SEQUENCE LISTING (20 sequences)

SEQ ID NO: 1 - protein, 16 aa, MUS SP.
RSSQSLGHSNGNTYLH

SEQ ID NO: 2 - protein, 16 aa, MUS SP.
RSSQSLVHSNGNTYLH

SEQ ID NO: 3 - protein, 16 aa, MUS SP.
  MISC_FEATURE (7)..(7): Xaa at position 7 = Gly, Ala, Val, Leu or Ile
RSSQSLXHSNGNTYLH

SEQ ID NO: 4 - protein, 7 aa, MUS SP.
KVSNRFS

SEQ ID NO: 5 - protein, 9 aa, MUS SP.
SQSTLVPWT

SEQ ID NO: 6 - protein, 10 aa, MUS SP.
GYTFTSYWMH

SEQ ID NO: 7 - protein, 10 aa, MUS SP.
GYTFTSYWIH

SEQ ID NO: 8 - protein, 10 aa, MUS SP.
  MISC_FEATURE (9)..(9): Xaa at position 9 = Met, Ile, Leu or Val
GYTFTSYWXH

SEQ ID NO: 9 - protein, 17 aa, MUS SP.
YINPSTGYTEYTQKFKD

SEQ ID NO: 10 - protein, 17 aa, MUS SP.
YIDPGIGNIEYNQKFQD

SEQ ID NO: 11 - protein, 18 aa, MUS SP.
  MISC_FEATURE (3)..(3): Xaa at position 3 = Asn, Gln, Asp or Glu
  MISC_FEATURE (5)..(5): Xaa at position 5 = Ser, Thr, Gly or Aala
  MISC_FEATURE (6)..(6): Xaa at position 6 is Thr, Ser, Ile, Leu or Val
  MISC_FEATURE (8)..(8): Xaa at position 8 = Tyr, Asn or Gln
  MISC_FEATURE (9)..(9): Xaa at position 9 = Thr, Ser, Ile, Leu or Val
  MISC_FEATURE (13)..(13): Xaa at position 13 = Thr, Ser, Asn or Gln
  MISC_FEATURE (17)..(17): Xaa at position 17 = Lys, Arg, Asn or Gln
YIXPXXGXXIEYXQKFXD

SEQ ID NO: 12 - protein, 7 aa, MUS SP.
DGYDEDY

SEQ ID NO: 13 - protein, 126 aa, MUS SP.
STPAWADAVMTQIPLTLSVTIGQPASISCRSSQSLGHSNGNTYLHWYLQKPGQSPKLLIYKVSNRFSGVPDRFSGSGSGTDFTLKISRVEAEDLGTYFCSQSTLVPWTFGGGTKLEIKRADAAPTV

SEQ ID NO: 14 - protein, 126 aa, MUS SP.
STPAWADVVMTQTPLSLPVSLGDQASISCRSSQSLVHSNGNTYLHWYLQKPGQSPKLLIYKVSNRFSGVPDRFSGSGSGTDFTLKISRVEAEDLGTYFCSQSTLVPWTFGGGTKLEIKRADAAPTV

SEQ ID NO: 15 - protein, 122 aa, MUS SP.
QVQLQQSRAELAKPGASVKMSCKASGYTFTSYWMHWVKQGPGQGLEWIGYINPSTGYTEYTQKFKDKATLTADKSSSTAYMQLSSLTSEDSAVYYCATDGYDEDYWGQGTTLTVSSAKTTPP

SEQ ID NO: 16 - protein, 122 aa, MUS SP.
QVQLQQSRAELAKPGASVKMSCKASGYTFTSYWIHWIKQRPGQGLEWIGYIDPGIGNIEYNQKFQDKATLTADKSSSIVYMQLNRLTSEDSAVYYCATDGYDEDYWGQGTTLTVSSAKTTPP

SEQ ID NO: 17 - DNA, 378 bp, MUS SP.
tctactccagcttgggcagatgttgtgatgacccaaactccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggaacttatttctgctctcaaagtacacttgttccgtggacgttcggtggaggcaccaagctggaaatcaagcgggctgatgctgcaccaactgta

SEQ ID NO: 18 - DNA, 366 bp, MUS SP.
caggtccagctgcagcagtctagggctgaactggcaaaacctggggcctcagtgaagatgtcctgcaaggcttctggctacacctttactagctactggatgcactgggtaaaacaggggcctggacagggtctggaatggattggatacattaatcctagcactggttatactgagtacactcagaagttcaaggacaaggccacattgactgcagacaaatcctccagcacagcctacatgcaactgagcagcctgacatctgaggactctgcagtctattactgtgcaacagatggttacgacgaggactactggggccaaggcaccactctcacagtctcctcagccaaaacgacaccccca

SEQ ID NO: 19 - protein, 28 aa, MUS SP.
  MISC_FEATURE (28)..(28): Xaa at position 28 = Arg or absent
GSSFLSPEHQRVQQRKESKKPPAKLQPX

SEQ ID NO: 20 - protein, 14 aa, MUS SP.
QRKESKKPPAKLQP

(19) United States
(12) Patent Application Publication
(10) Pub. No.: US 2007/0237775 A1
(43) Pub. Date: Oct. 11, 2007

(54) Anti-Ghrelin Antibodies

(75) Inventors: Kristine Kay Kikly, Fortville, IN (US); Joseph Vincent Manetta, Indianapolis, IN (US); Derrick Ryan Witcher, Fishers, IN (US)

Correspondence Address: ELI LILLY & COMPANY, PATENT DIVISION, P.O. BOX 6288, INDIANAPOLIS, IN, 46206-6288, US

(73) Assignee: Eli Lilly and Company, Indianapolis, IN (US)

(21) Appl. No.: 11/569,769

(22) PCT Filed: Jul. 6, 2005
(86) PCT No.: PCT/US05/23968

§ 371 Date: Nov. 29, 2006

Related U.S. Application Data
(60) Provisional application No. 60/587,681, filed on Jul. 14, 2004.

(51) Int. Cl. A61K 39/395; C07K 16/26

(52) U.S. Cl. 424/141.1; 530/388.1

(57) ABSTRACT

Monoclonal antibodies, including chimeric and humanized antibodies, that bind both acylated and unacylated human ghrelin are disclosed. An antibody of the invention can be a full-length antibody or an antigen-binding portion thereof. Such antibodies and antigen-binding portions thereof are useful for neutralizing ghrelin activity in, for example, a human subject suffering from a disorder in which ghrelin activity is detrimental.

### FIELD OF THE INVENTION

The present invention is in the field of medicine, particularly in the field of monoclonal antibodies against human ghrelin. More specifically the invention relates to monoclonal antibodies that specifically bind both the acylated and unacylated forms of human ghrelin. The antibodies of the invention bind an antigenic epitope located within amino acids 14-27 of human ghrelin and are useful for treatment of various diseases or disorders in mammals wherein a decrease in ghrelin level or activity contributes to a desirable therapeutic effect, e.g., obesity and obesity-related disorders such as NIDDM.

### BACKGROUND OF THE INVENTION

Ghrelin is a 28 amino acid peptide, a portion of which is acylated, typically with an n-octanoyl group, at the amino acid at position three (see SEQ ID NO: 19). The ghrelin hormone, when acylated, binds the growth hormone secretagogue receptor (GHS-R1a) in the pituitary thereby stimulating release of growth hormone. Acylated ghrelin is also involved in, e.g., energy balance, gastric motility and anxiety (Masuda, et al., Biochem Biophy Res Commun, 276:905-908, 2000; Asakawa, A. et al., Neuroendocrinology, 74:143-147, 2001). The acylated form of ghrelin furthermore leads to fat deposition when administered to mice (Tschop, M. et al., Nature 407:908-913, 2000).

The unacylated or “des-acyl” form of ghrelin, does not bind the GHS-R1a receptor (Kojima, M. et al., Nature 402:656-660, 1999). It has been demonstrated that des-acyl ghrelin, present in the bloodstream at 2.5-fold greater concentration than acylated ghrelin, is not without biological activity. des-acyl ghrelin shares with acylated ghrelin some non-endocrine actions like cardiovascular effects, modulation of cell proliferation and some influence on adipogenesis (Broglio, F. et al., J. Clin. Endo & Met., 89:3062-3065, 2004). Des-acyl ghrelin may bind an as-yet unidentified GHS-R subtype.

Ghrelin is synthesized primarily in the stomach and circulated in the blood. Ghrelin serum levels increase during food deprivation in animals peak prior to eating and decrease upon refeeding (Kojima, M. et al., Nature 402:656-660, 1999, Cummings, et al., New Eng. J. Med., 346:1623-1630, 2002). It has been shown that persons who underwent gastric bypass surgery and lost up to 36% of their body weight had greatly reduced circulating ghrelin levels and loss of pre-meal peaks in ghrelin secretion. Persons with Prader-Willi syndrome, a genetic disorder that causes severe obesity with uncontrollable appetite, have extremely high levels of ghrelin (Cummings, et al., supra). These observations indicate that ghrelin plays a key role in motivating feeding. Additionally, ghrelin is believed to signal the hypothalamus when an increase in metabolic efficiency is required. (Muller, et al., Clin Endocrinol. 55:461-467, 2001). Numerous ghrelin review articles are available, e.g., van der Lely, A., et al., Endocrine Reviews 25:426-457, 2004.

International patent publication number WO 01/07475 (EP1197496) teaches the ghrelin amino acid sequence of various species, including human, and discloses that a portion of the ghrelin population is acylated, typically with O-n-octanoic acid, at the third amino acid from the amino terminus, which is serine in native human ghrelin. WO 01/07475 also indicates that the amino terminal four amino acids of acylated ghrelin are essential for the GHSR1a receptor binding activity of acylated ghrelin. The application further teaches antibodies directed against fatty acid-modified peptides of ghrelin, which peptides induce signal transduction, and the use of such antibodies for assaying or detecting ghrelin.

International patent publication number WO 01/87335 teaches the use of agents that specifically bind ghrelin, including anti-ghrelin antibodies, for the treatment of obesity.

Provisional patent application numbers (i) 60/475,708 filed Jun. 4, 2003; (ii) 60/491,352 filed Jul. 31, 2003, and (iii) 60/501,465 filed Sep. 9, 2003 all entitled “Anti-Ghrelin Antibodies” and assigned to Eli Lilly and Company, teach monoclonal anti-ghrelin antibodies which preferentially bind acylated human ghrelin (at an epitope localized within amino acids 1-8 of acylated human ghrelin) with respect to unacylated human ghrelin and are useful for treatment of obesity and obesity-related disorders.

Provisional patent application numbers (i) 60/500,496 filed Sep. 5, 2003; (ii) 60/572,249 filed Mar. 18, 2004, and (iii) a third filed Jul. 23, 2004, all entitled “Anti-Ghrelin Antibodies” and assigned to Eli Lilly and Company, teach monoclonal anti-ghrelin antibodies which bind both the acylated and unacylated forms of human ghrelin at an epitope localized within amino acids 4-20 of human ghrelin.

International patent publication number WO 03/051389 teaches that administration of des-acyl ghrelin may prevent or reduce postprandial induction of insulin resistance by antagonizing some ghrelin actions and may reduce body weight in some patients.

Murakami, N. et al., administered to obese rats by intracerebroventricular injection, a polyclonal anti-ghrelin antibody raised against the acylated amino-terminal eleven amino acids of rat ghrelin. The authors were able to demonstrate a subsequent decrease in both food intake and body weight by the rats. J. Endocrinology 174:283-288, 2002.

Obesity is a complex, chronic disease characterized by excessive accumulation of body fat and has a strong familial component. Obesity is generally the result of a combination of factors including genetic factors. Approximately 6% of the total population of the United States is morbidly obese. Morbid obesity is defined as having a body mass index of more than forty, or, as is more commonly understood, being more than one hundred pounds overweight for a person of average height. Obesity is related to other disorders and diseases, i.e., obesity increases the risk of illness from about 30 serious medical conditions including osteoarthritis, Type II diabetes, hypertension, cancer and cardiovascular disease, and is associated with increases in deaths from all causes. Additionally, obesity is associated with depression and can further affect the quality of life through limited mobility and decreased physical endurance.

There are presently limited treatments for obesity. Current treatment options to manage weight include dietary therapy, increased physical activity and behavior therapy. Unfortunately, these treatments are largely unsuccessful with a failure rate reaching 95%. This failure may be due to the fact that the condition is strongly associated with genetically inherited factors that contribute to increased appetite, preference for highly caloric foods, reduced physical activity and increased lipogenic metabolism. This indicates that people inheriting these genetic traits are prone to becoming obese regardless of their efforts to combat the condition. Gastric bypass surgery is available to a limited number of obese persons. However, this type of surgery involves a major operation and cannot be modified readily as patient needs change. Additionally, even this attempted remedy can sometimes fail (see, e.g., Kriwanek, Langenbecks Archiv. Fur Chirurgie, 38:70-74, 1995). Drug therapy options are few and of limited utility. Moreover, chronic use of these drugs can lead to tolerance, as well as side effects from long-term administration. And, when the drug is discontinued, weight often returns.

There is a tremendous therapeutic need for a means to treat obesity, obesity-related disorders and diseases, as well as other eating disorders and disorders which correlate with elevated ghrelin levels. Due to its role in inducing feeding, ghrelin is a desirable target for therapeutic intervention. In particular, a monoclonal antibody against ghrelin may provide such a therapy. Of particular importance therapeutically is a humanized form of such a monoclonal antibody. Additionally, ghrelin is highly conserved in sequence and in function across species; therefore, not only may a monoclonal antibody of the invention be useful for the treatment of ghrelin-associated disorders in humans, but also in other mammals including, e.g., domestic animals (e.g., canine and feline), sports animals (e.g., equine) and food-source animals (e.g., bovine, porcine and ovine) and laboratory animals (e.g. rat). An anti-ghrelin monoclonal antibody of the invention may be useful for the treatment or prevention of obesity, obesity-related disorders, NIDDM (Type II diabetes), Prader-Willi syndrome, eating disorders, hyperphagia, impaired satiety, anxiety, gastric motility disorders (including e.g., irritable bowel syndrome and functional dyspepsia), insulin resistance syndrome, metabolic syndrome, dyslipidemia, atherosclerosis, hypertension, hyperandrogenism, polycystic ovarian syndrome, cancer, and cardiovascular disorders. Additionally, an anti-ghrelin monoclonal antibody of the invention may be useful for the treatment or prevention of any disease or disorder which benefits from lower levels or lower activity of either the acylated or unacylated forms of ghrelin or both.

### SUMMARY OF THE INVENTION

Monoclonal antibodies against human ghrelin (“hGhrelin”) that specifically bind an epitope localized within an antigenic peptide spanning amino acids 14-27 (inclusive) common to both the acylated and unacylated (“des-acyl”) forms of hGhrelin (i.e., QRKESKKPPAKLQP, SEQ ID NO: 20) are described in the present invention. Such antibodies are referred to herein as “anti-hGhrelin monoclonal antibodies” or “antibodies of the invention” or “monoclonal antibodies of the invention.” The monoclonal antibodies of the invention may specifically bind a peptide with the sequence shown in SEQ ID NO: 20 when it is located within acylated or des-acyl ghrelin or when it is independent of any additional ghrelin sequence. The monoclonal antibodies of the invention include murine monoclonal antibodies as well as chimeric monoclonal antibodies and humanized monoclonal antibodies and antigen-binding fragments thereof. Preferably the antibodies of the invention exist in a homogeneous or substantially homogeneous population.

Preferably the antibodies of the invention specifically bind acylated hGhrelin with no greater than six-fold or five-fold; more preferably no greater than four-fold or three-fold, and most preferably no greater than two-fold difference than with which they specifically bind des-acyl hGhrelin as determined using available laboratory techniques, e.g., by ELISA assay or by K_{D} values in a Biacore™ assay. The antibodies of the invention disrupt or antagonize at least one in vitro or in vivo or in situ bioactivity or biological property associated with acylated or des-acyl hGhrelin or both.

Preferably the monoclonal antibodies of the invention have a k_{off} value less than 10⁻³, 10⁻⁴, 10⁻⁵ and more preferably less than 10⁻⁶ or less than 10⁻⁷ (1/sec). Preferably the monoclonal antibodies of the invention have a kₒₙ value of greater than 10⁵ or 10⁶ and more preferably greater than 10⁷ (1/Msec). Preferably the monoclonal antibodies of the invention have a K_{D} value of less than 10⁻⁹ or 10⁻¹⁰, and more preferably less than 10⁻¹¹ or 10⁻¹² (M).

The invention provides an “antigenic peptide” which comprises, or alternatively consists of, an antigenic epitope, to which antibodies of the invention specifically bind. The antigenic peptide spans 14, 13, 12, 11, 10, 9, 8, 7 or 6 contiguous amino acids of human ghrelin and is localized within the peptide spanning amino acids 14-27 (inclusive) of human ghrelin. The antigenic peptide may exist independently or be conjugated to a non-ghrelin peptide, e.g., a immune potentiator, e.g., keyhole limpet hemocyanin (KLH), through an amino acid, preferably a cysteine residue, added to the C-terminus of the antigenic peptide. The antigenic peptide may be used to administer to non-human animals to generate monoclonal antibodies of the invention.

In one embodiment, an anti-hGhrelin monoclonal antibody of the invention comprises at least 1 or 2, more preferably 3, 4 or 5 peptides from peptides with a sequence selected from the group consisting of (a) SEQ ID NO: 1, 2, or 3; (b) SEQ ID NO: 4; (c) SEQ ID NO: 5; (d) SEQ ID NO: 6, 7 or 8; (e) SEQ ID NO: 9, 10 or 11; and (f) SEQ ID NO: 12. Preferably, the peptide with the sequence shown in SEQ ID NO: 1, 2, or 3, when present in an antibody of the invention, is at light chain variable region (“LCVR”) CDR1. Preferably the peptide with the sequence shown in SEQ ID NO: 4, when present in an antibody of the invention, is at LCVR CDR2. Preferably the peptide with the sequence shown in SEQ ID NO: 5, when present in an antibody of the invention, is at LCVR CDR3. Preferably the peptide with the sequence shown in SEQ ID NO: 6, 7 or 8, when present in an antibody of the invention, is at heavy chain variable region (“HCVR”) CDR1. Preferably the peptide with the sequence shown in SEQ ID NO: 9, 10 or 11, when present in an antibody of the invention, is at HCVR CDR2. Preferably the peptide with the sequence shown in SEQ ID NO: 12, when present in an antibody of the invention, is at HCVR CDR3. For approximate CDR locations within the LCVR or HCVR, see Table 2 herein or SEQ ID NOs: 13-16.

One embodiment provides an anti-hGhrelin monoclonal antibody comprising the 6 peptides with the sequences shown in SEQ ID NOs: 1, 4, 5, 6, 9 and 12. Preferably, the peptide with the sequence shown in SEQ ID NO: 1 is located at LCVR CDR1, the peptide with the sequence shown in SEQ ID NO: 4 is located at LCVR CDR2, the peptide with the sequence shown in SEQ ID NO: 5 is located at LCVR CDR3, the peptide with the sequence shown in SEQ ID NO: 6 is located at HCVR CDR1, the peptide with the sequence shown in SEQ ID NO: 9 is located at HCVR CDR2, and the peptide with the sequence shown in SEQ ID NO: 12 is located at HCVR CDR3. (See 3281 in Table 1).

Another embodiment provides an anti-hGhrelin monoclonal antibody comprising the 6 peptides with the sequences as shown in SEQ ID NOs: 2, 4, 5, 6, 9 and 12. Preferably, the peptide with SEQ ID NO: 2 is located at LCVR CDR1, the peptide with SEQ ID NO: 4 is located at LCVR CDR2, the peptide with SEQ ID NO: 5 is located at LCVR CDR3, the peptide with SEQ ID NO: 6 is located at HCVR CDR1, the peptide with SEQ ID NO: 9 is located at HCVR CDR2, and the peptide with SEQ ID NO: 12 is located at HCVR CDR3. (See 4731 in Table 1).

Another embodiment provides an anti-hGhrelin monoclonal antibody comprising the 6 peptides with the sequences as shown in SEQ ID NOs: 2, 4, 5, 7, 10 and 12. Preferably, the peptide with SEQ ID NO: 2 is located at LCVR CDR1, the peptide with SEQ ID NO: 4 is located at LCVR CDR2, the peptide with SEQ ID NO: 5 is located at LCVR CDR3, the peptide with SEQ ID NO: 7 is located at HCVR CDR1, the peptide with SEQ ID NO: 10 is located at HCVR CDR2, and the peptide with SEQ ID NO: 12 is located at HCVR CDR3. (See 4281 in Table 1).

Another embodiment provides an anti-hGhrelin monoclonal antibody comprising the 6 peptides with the sequences as shown in SEQ ID NOs: 3, 4, 5, 8, 11 and 12. Preferably, the peptide with SEQ ID NO: 3 is located at LCVR CDR1, the peptide with SEQ ID NO: 4 is located at LCVR CDR2, the peptide with SEQ ID NO: 5 is located at LCVR CDR3, the peptide with SEQ ID NO: 8 is located at HCVR CDR1, the peptide with SEQ ID NO: 11 is located at HCVR CDR2, and the peptide with SEQ ID NO: 12 is located at HCVR CDR3. (See consensus in Table 1).

In another embodiment, an anti-hGhrelin monoclonal antibody of the invention comprises a light chain variable region (LCVR) comprising a peptide with the sequence shown in SEQ ID NO: 13 or 14. In another embodiment, an anti-hGhrelin monoclonal antibody of the invention comprises a heavy chain variable region (HCVR) comprising a peptide with the sequence shown in SEQ ID NO: 15 or 16. In another embodiment, an anti-hGhrelin monoclonal antibody of the invention comprises a LCVR comprising a peptide with the sequence shown in SEQ ID NO: 13 or 14 and further comprises a HCVR comprising a peptide with the sequence shown in SEQ ID NO: 15 or 16. An anti-hGhrelin monoclonal antibody of the invention may comprise a LCVR comprising a peptide with the sequence shown in SEQ ID NO: 13 and further comprise a HCVR comprising a peptide with the sequence shown in SEQ ID NO: 15. An anti-hGhrelin monoclonal antibody of the invention may comprise a LCVR comprising a peptide with the sequence shown in SEQ ID NO: 14 and further comprise a HCVR comprising a peptide with the sequence shown in SEQ ID NO: 15 or 16.

Preferably the LCVR CDR1 of an anti-hGhrelin monoclonal antibody of the invention comprises a peptide with the sequence shown in SEQ ID NO: 1, 2 or 3. Preferably the LCVR CDR2 of an anti-hGhrelin monoclonal antibody of the invention comprises a peptide with the sequence shown in SEQ ID NO: 4. Preferably the LCVR CDR3 of an anti-hGhrelin monoclonal antibody of the invention comprises a peptide with the sequence shown in SEQ ID NO: 5. Preferably the HCVR CDR1 of an anti-hGhrelin monoclonal antibody of the invention comprises a peptide with the sequence shown in SEQ ID NO: 6, 7 or 8. Preferably the HCVR CDR2 of an anti-hGhrelin monoclonal antibody of the invention comprises a peptide with the sequence shown in SEQ ID NO: 9, 10, or 11. Preferably the HCVR CDR3 of an anti-hGhrelin monoclonal antibody of the invention comprises a peptide with the sequence shown in SEQ ID NO: 12.

The invention further embodies an antibody which competitively inhibits in vivo or in vitro binding of any of the antibodies 3281, 4731 or 4281 as measured by any method known in the art, preferably competitive ELISA assay or BLAcore™ assay or FLIPR assay as described, e.g., in the Examples herein, or by Western blot, immunoprecipitation or FACS.

An anti-hGhrelin monoclonal antibody of the invention may further comprise a heavy chain constant region selected from the group consisting of IgG₁, IgG₂, IgG₃, IgG₄, IgA, IgE, IgM and IgD. Preferably the heavy chain constant region is IgG₄ or IgG₁. An anti-hGhrelin monoclonal antibody of the invention may further comprise a kappa or lambda chain constant region.

An anti-hGhrelin monoclonal antibody of the invention may comprise, or consist of, an intact antibody (i.e., full length), a substantially intact antibody, a Fab fragment, a F(ab′)₂ fragment, a single chain Fv fragment, or any antigen binding (i.e., “antigenic peptide” binding) fragment thereof.

An anti-hGhrelin monoclonal antibody of the invention may comprise 1, 2, 3, 4, 5 or 6 peptides selected from peptides with a sequence selected from the group consisting of: (a) SEQ ID NO: 1, 2 or 3 at LCVR CDR1; (b) SEQ ID NO: 4 at LCVR CDR2; (c) SEQ ID NO: 5 at LCVR CDR3; (d) SEQ ID NO: 6, 7 or 8 at HCVR CDR1; (e) SEQ ID NO: 9, 10, or 11 at HCVR CDR2; and (f) SEQ ID NO: 12 at HCVR CDR3; in which said peptide has 2 or 1 conservative amino acid substitutions and/or terminal deletions with respect to the sequence shown in said SEQ ID Number.

In a preferred embodiment, an anti-hGhrelin monoclonal antibody of the invention is a chimeric antibody. In a more preferred embodiment, an anti-hGhrelin monoclonal antibody of the invention is a humanized antibody in which framework sequence and constant region present in the antibody is of human origin or substantially of human origin. The humanized antibody is preferably a full-length antibody. Alternatively, the framework region, or a portion thereof, and constant region present in the antibody may substantially originate from the genome of the animal in which the antibody is to be used as a therapeutic (e.g., canine, feline, equine, bovine, porcine and ovine).

In another embodiment, the invention provides an isolated nucleic acid molecule comprising a DNA molecule encoding a polypeptide comprising an LCVR of an antibody of the invention, and/or a polypeptide comprising an HCVR of an antibody of the invention (i.e., “nucleic acid molecule of the invention”). In an exemplary embodiment, the polypeptide comprising an LCVR of an antibody of the invention (e.g., 4731) is encoded by a polynucleotide comprising the sequence shown in SEQ ID NO: 17. In another embodiment, the polypeptide comprising an HCVR of an antibody of the invention (e.g., 4731) is encoded by a polynucleotide comprising the sequence shown in SEQ ID NO: 18.

In another embodiment, the invention provides a vector, preferably a plasmid, a recombinant expression vector, a yeast expression vector or a retroviral expression vector, comprising a polynucleotide encoding a polypeptide comprising an anti-hGhrelin monoclonal antibody of the invention or an antigen-binding fragment thereof. Alternatively, a vector of the invention comprises a polynucleotide encoding a polypeptide comprising a LCVR and/or a HCVR present in an anti-hGhrelin monoclonal antibody of the invention. By way of example, the vector of the invention may comprise a polynucleotide comprising the sequence shown in SEQ ID NO: 17 and/or a polynucleotide comprising the sequence shown in SEQ ID NO: 18.

When a polynucleotide encoding a polypeptide comprising a LCVR of an antibody of the invention and a polynucleotide encoding a polypeptide comprising a HCVR of an antibody of the invention are present in one vector, the LCVR and HCVR sequence may be transcribed from one promoter to which they are both operably linked; or they may be transcribed independently, each from a separate promoter to which it is operably linked. If the DNA sequences encoding said LCVR and HCVR are present in the same vector and transcribed from one promoter to which they are both operably linked, the LCVR sequence may be 5′ to the HCVR sequence or the LCVR sequence may be 3′ to the HCVR sequence, furthermore the LCVR and HCVR coding region in the vector may be separated by a linker sequence of any size or content, preferably such linker, when present, is a polynucleotide comprising an internal ribosome entry site.

In another embodiment, the invention provides a host cell comprising a nucleic acid molecule of the present invention. Preferably a “host cell of the invention” comprises one or more vectors or constructs comprising a nucleic acid molecule of the present invention. The host cell of the invention is a cell into which a vector of the invention has been introduced (e.g., via transformation, transduction, infection and the like). The invention also provides a host cell into which two vectors of the invention have been introduced; one comprising a polynucleotide encoding a polypeptide comprising a LCVR present in an antibody of the invention and one comprising a polynucleotide encoding a polypeptide comprising a HCVR present in an antibody of the invention and preferably, each LCVR and HCVR coding region is operably linked to a promoter sequence. Preferably the vectors are integrated into the chromosomal DNA of the host cell. The host cell types include mammalian, bacterial, plant and yeast cells. Preferably the host cell is a CHO cell, CHO-K1 cell, COS cell, SP2/0 cell, NS0 cell, yeast cell or a derivative or progeny of a preferred cell type.

In another embodiment, the invention provides a method of synthesizing an anti-hGhrelin monoclonal antibody of the invention comprising culturing a host cell of the invention in culture media such that an anti-hGhrelin monoclonal antibody of the invention or an antigen-binding fragment thereof is expressed in the cell. The antibody (or antigen-binding fragment thereof) is purified from the host cell or preferably from the culture media in which said host cell is grown.

The invention further embodies the process of producing an antibody of the invention by (i) immunizing a non-human animal, preferably a mouse or rat, with an immunogenic peptide comprising, or consisting of, 14, 13, 12, 11, 10, 9, 8, 7 or 6 contiguous amino acids of the peptide spanning amino acid residues 14-27 of human ghrelin (see SEQ ID NO: 18) wherein the immunogenic peptide is optionally conjugated to an immune potentiator, and (ii) identifying and isolating a monoclonal antibody from the immunized animal using any method known in the art, preferably by hybridoma synthesis. The anti-ghrelin antibodies are screened by any method available in the art (e.g., phage display, ribosome display, yeast display, bacterial display, ELISA assay) to identify an antibody that specifically binds both acylated hGhrelin and des-acyl hGhrelin at an antigenic epitope located within amino acids 14-27 of human ghrelin. The invention further embodies a monoclonal antibody made by this process. Preferably said monoclonal antibody binds acylated hGhrelin with no greater than six-fold or five-fold; more preferably no greater than four-fold or three-fold, and most preferably no greater than two fold difference than with which it binds des-acyl hGhrelin as determined by any art-known method, e.g., by ELISA assay or K_{D} values in a BLAcore™ assay. It is contemplated that said antibody may be further altered into a chimeric antibody or a humanized antibody, using methods known in the art, and still fall within the scope of the invention.

Various forms of the antibodies of the invention are contemplated herein. For example, an anti-hGhrelin monoclonal antibody of the invention may be a full length antibody (e.g., having a murine or, preferably, human immunoglobulin constant region) or any antigen-binding fragment thereof (e.g., a F(ab′)₂). It is understood that all such forms of the antibodies are encompassed herein within the term “antibody.” Furthermore, the antibody may be labeled with a detectable label, immobilized on a solid phase and/or conjugated with a heterologous compound (e.g., an enzyme or toxin or other detectable label e.g., radiolabel, chromophore, fluorescer) according to methods known in the art.

Diagnostic uses for antibodies of the invention are contemplated. In one diagnostic application, the invention provides a method for determining the presence of ghrelin protein comprising exposing a test sample suspected of containing the ghrelin protein to an anti-hGhrelin antibody of the invention and determining specific binding of the antibody to the sample. An anti-hGhrelin antibody of the invention may be used to determine the levels of ghrelin in test samples by comparing test sample values to a standard curve generated by binding said antibody to samples with known amounts of ghrelin. For diagnostic use, the invention provides a kit comprising an antibody of the invention and instructions for using the antibody to detect ghrelin protein in, e.g., a test sample.

In another embodiment, the invention provides a pharmaceutical composition comprising an anti-hGhrelin monoclonal antibody of the invention. The pharmaceutical composition of the invention may further comprise a pharmaceutically acceptable carrier. In said pharmaceutical composition, the anti-hGhrelin monoclonal antibody of the invention is the active ingredient. Preferably the pharmaceutical composition comprises a homogeneous or substantially homogeneous population of an anti-hGhrelin monoclonal antibody of the invention. The composition for therapeutic use is sterile and may be lyophilized.

The invention provides a method of inhibiting ghrelin activity or decreasing active ghrelin levels in a subject, preferably a human, in need thereof, whether that activity results from acylated ghrelin or des-acyl ghrelin or both, comprising administering a therapeutically effective amount, or prophylactically effective amount, of an anti-hGhrelin monoclonal antibody of the invention to said subject. The invention further provides a method of treating or preventing a disease or disorder ameliorated by the inhibition of signal transduction resulting from the binding of ghrelin to GHS-R1a which comprises administering to a subject or patient (e.g., a human), in need of such treatment or prevention, a therapeutically or prophylactically effective amount of a monoclonal antibody of the invention. As used herein, the term “disease or disorder ameliorated by inhibition of signal transduction resulting from the binding of ghrelin to GHS-R1a” means conditions associated with abnormal ghrelin levels or benefited by a change in the existing ghrelin level, whether it be acylated ghrelin or des-acyl ghrelin. Diseases or disorders treated or prevented with a monoclonal antibody of the invention include, but are not limited to, obesity, obesity-related disorders, NIDDM (Type II diabetes), Prader-Willi syndrome, eating disorders, hyperphagia, impaired satiety, anxiety, gastric motility disorders (including e.g., irritable bowel syndrome and functional dyspepsia), insulin resistance syndrome, metabolic syndrome, dyslipidemia, atherosclerosis, hypertension, hyperandrogenism, polycystic ovarian syndrome, cancer, and cardiovascular disorders in a subject, e.g., a human.

The invention embodies an anti-hGhrelin monoclonal antibody of the invention for use in the manufacture of a medicament for administration to a subject, e.g., a human, in need thereof for the treatment of obesity, obesity-related disorders, NIDDM (Type II diabetes), Prader-Willi syndrome, eating disorders, hyperphagia, impaired satiety, anxiety, gastric motility disorders (including e.g., irritable bowel syndrome and functional dyspepsia), insulin resistance syndrome, metabolic syndrome, dyslipidemia, atherosclerosis, hypertension, hyperandrogenism, polycystic ovarian syndrome, cancer, and cardiovascular disorders.

The invention further embodies an anti-hGhrelin monoclonal antibody of the invention for use in the manufacture of a medicament for administration to other mammals including domestic animals, food source animals, sports animals and laboratory animals for the prevention or treatment of the disorders listed above.

The invention embodies an article of manufacture comprising a packaging material and a monoclonal antibody of the invention contained within said packaging material and wherein the packaging material comprises a package insert which indicates that the antibody neutralizes a ghrelin activity or decreases the level of active ghrelin.

TABLE 1
CDR Sequences of Fabs 3281, 4731 and 4281

| | | |
|---|---|---|
| CDR1 | CDR2 | CDR3 |

| | | | |
|---|---|---|---|
| Light Chain | | | |
| 3281 | RSSQSLGHSNGNTYLH | KVSNRFS | SQSTLVPWT |
| LCVR | (SEQ ID NO: 1) | (SEQ ID NO: 4) | (SEQ ID NO: 5) |
| 4731 | RSSQSLVHSNGNTYLH | KVSNRFS | SQSTLVPWT |
| LCVR | (SEQ ID NO: 2) | (SEQ ID NO: 4) | (SEQ ID NO: 5) |
| 4281 | RSSQSLVHSNGNTYLH | KVSNRFS | SQSTLVPWT |
| LCVR | (SEQ ID NO: 2) | (SEQ ID NO: 4) | (SEQ ID NO: 5) |
| Consensus | RSSQSLX₇HSNGNTYLH | KVSNRFS | SQSTLVPWT |
| | (SEQ ID NO: 3) | (SEQ ID NO: 4) | (SEQ ID NO: 5) |
| Heavy Chain | | | |
| 3281 | GYTFTSYWMH | YINPSTGYTEYTQKFKD | DGYDEDY |
| HCVR | (SEQ ID NO: 6) | (SEQ ID NO: 9) | (SEQ ID NO: 12) |
| 4731 | GYTFTSYWMH | YINPSTGYTEYTQKFKD | DGYDEDY |
| HCVR | (SEQ ID NO: 6) | (SEQ ID NO: 9) | (SEQ ID NO: 12) |
| 4281 | GYTFTSYWIH | YIDPGIGNIEYNQKFQD | DGYDEDY |
| HCVR | (SEQ ID NO: 7) | (SEQ ID NO: 10) | (SEQ ID NO: 12) |
| Consensus | GYTFTSYWX₉H | YIX₃PX₅X₆GX₈X₉IEYX₁₃QKFX₁₇D | DGYDEDY |
| HCVR | (SEQ ID NO: 8) | (SEQ ID NO: 11) | |

### DETAILED DESCRIPTION OF THE INVENTION

The invention provides anti-ghrelin antibodies (including antigen-binding fragments thereof) which are capable of specifically binding to human ghrelin at an epitope localized to (i.e., falls within) amino acids 14-27 of human ghrelin. Preferred anti-ghrelin antibodies are capable of modulating a biological activity associated with ghrelin, and thus are useful in the treatment or prevention of various diseases and pathological conditions, including obesity and obesity related diseases.

One active form of ghrelin present in humans is a 28 amino acid peptide (SEQ ID NO: 19) acylated, typically with an n-octanoyl group, at the serine amino acid located at position 3. Acylated ghrelin was identified as the endogenous ligand of the growth hormone secretagogue receptor 1a (GHS-R1a) (Kojima, M. et al. Nature 402:656-660, 1999). It is secreted from multiple organs of the body but primarily from the stomach. The unacylated, or “des-acyl” form of ghrelin does not bind GHS-R1a but likely binds another subtype of the GHS-R.

Recently ghrelin peptides with various modifications of the predominant form of ghrelin (SEQ ID NO: 19) have been identified in human stomach (Hosoda, H. et al., J. Biol. Chem. 278:64-70, 2003). These minor forms include a 27 amino acid ghrelin peptide lacking the C-terminal Arg of the sequence that is shown in SEQ ID NO: 19 and ghrelin peptides decanoylated or decenoylated at position 3. The antibodies of the present invention bind both the 28 and 27 amino acid forms of hGhrelin (or even shorter forms when C-terminal deleted) both in the acylated and des-acyl form.

When it is necessary herein to refer specifically to the acylated form of ghrelin, it is referred to herein as “acylated ghrelin,” or “acylated hGhrelin” when referring specifically to human ghrelin. When referring herein specifically to the unacylated form of ghrelin, the term “des-acyl ghrelin” or “des-acyl hGhrelin” is used herein.

Antibodies are typically proteins or polypeptides which exhibit binding specificity to a specific antigen. A full-length antibody as it exists naturally is an immunoglobulin molecule comprised of four peptide chains, two identical heavy (H) chains (about 50-70 kDa when full length) and two identical light (L) chains (about 25 kDa when full length) interconnected by disulfide bonds. The amino terminal portion of each chain includes a variable region of about 100-110 or more amino acids primarily responsible for antigen recognition. The carboxy-terminal portion of each chain defines a constant region primarily responsible for effector function.

Light chains are classified as kappa or lambda and characterized by a particular constant region. Heavy chains are classified as gamma, mu, alpha, delta, or epsilon, and define the antibody's isotype as IgG, IgM, IgA, IgD, and IgE, respectively. Each heavy chain type is characterized by a particular constant region.

Each heavy chain is comprised of a heavy chain variable region (herein “HCVR”) and a heavy chain constant region. The heavy chain constant region is comprised of three domains (CH1, CH2, and CH3) for IgG, IgD, and IgA; and 4 domains (CH1, CH2, CH3, and CH4) for IgM and IgE. Each light chain is comprised of a light chain variable region (herein “LCVR”) and a light chain constant region. The light chain constant region is comprised of one domain, CL. The HCVR and LCVR regions can be further subdivided into regions of hypervariability, termed complementarity determining regions (CDRs), interspersed with regions that are more conserved, termed framework regions (FR). Each HCVR and LCVR is composed of three CDRs and four FRs, arranged from amino-terminus to carboxy-terminus in the following order: FR1, CDR1, FR2, CDR2, FR3, CDR3, FR4. The assignment of amino acids to each domain is in accordance with well-known conventions [e.g., Kabat, “Sequences of Proteins of Immunological Interest,” National Institutes of Health, Bethesda, Md. (1987 and 1991) or Chothia numbering scheme as described in Al-Lazikani et al., J. Mol. Biol. 273:927-948, 1997, see also the internet site http:www.rubic.rdg.ac.uk/˜andrew/bioinforg/abs. The functional ability of an antibody to bind a particular antigen is determined collectively by the six CDRs. However, even a single variable domain comprising only three CDRs specific for an antigen may have the ability to recognize and bind antigen, although at a lower affinity than a complete Fab.

The term “antibody,” in reference to an anti-hGhrelin antibody of the invention (or simply, “antibody of the invention”), as used herein, refers to a monoclonal antibody. A “monoclonal antibody” as used herein refers to a murine monoclonal antibody, a chimeric antibody or a humanized antibody. The term “monoclonal antibody” as used herein is not limited to antibodies produced through hybridoma technology. The term “monoclonal antibody” as used herein refers to an antibody that is derived from a single copy or clone, including, e.g., any eukaryotic, prokaryotic, or phage clone, and not the method by which it is produced. A “monoclonal antibody” as used herein can be an intact (complete or full length) antibody, a substantially intact antibody, a portion or fragment of an antibody comprising an antigen-binding portion, e.g., a Fab fragment, Fab′ fragment or F(ab′)₂ fragment of a murine antibody or of a chimeric antibody or of a humanized antibody.

As used herein, the “antigen-binding portion” or “antigen-binding fragment” refers to a portion of an antibody molecule which contains amino acid residues that interact with an antigen and confer on the antibody its specificity and affinity for the antigen. This antibody portion includes the “framework” amino acid residues necessary to maintain the proper conformation of the antigen-binding residues. Preferably, the CDRs of the antigen-binding region of the monoclonal antibodies of the invention will be of murine origin or substantially of murine origin. In other embodiments, the antigen-binding region can be derived from other non-human species such as rabbit, rat or hamster. Examples of antibody fragments include Fab, Fab′, F(ab′)2, and Fv fragments, diabodies, single chain antibody molecules, and multispecific antibodies formed from antibody fragments.

Furthermore, a “monoclonal antibody” as used herein can be a single chain Fv fragment that may be produced by joining the DNA encoding the LCVR and HCVR with a linker sequence. (See, Pluckthun, The Pharmacology of Monoclonal Antibodies, vol. 113, Rosenburg and Moore eds., Springer-Verlag, New York, pp 269-315, 1994). It is understood that regardless of whether fragments are specified, the term “antibody” as used herein includes such antigen-binding fragments as well as single chain forms. As long as the protein retains the ability to specifically bind its intended target (e.g., epitope or antigen), it is included within the term “antibody.” Antibodies may or may not be glycosylated and fall within the bounds of the invention.

A “monoclonal antibody” as used herein when referring to a population of antibodies, refers to a homogeneous or substantially homogeneous (or pure) antibody population (i.e., at least about 90%, 92%, 95%, 96%, more preferably at least about 97% or 98% or most preferably at least 99% of the antibodies in the population are identical and would compete in an ELISA assay for the same antigen. A monoclonal antibody of the invention may be expressed by a hybridoma, expressed recombinantly, or synthesized synthetically by means readily known in the art. The monoclonal antibodies herein include chimeric, hybrid and recombinant antibodies produced by splicing a variable (including hypervariable) domain of an anti-ghrelin antibody with a constant domain (e.g. “humanized” antibodies), or a light chain with a heavy chain, or a chain from one species with a chain from another species, or fusions with heterologous proteins, regardless of species of origin or immunoglobulin class or subclass designation, as well as antibody fragments (e.g., Fab, F(ab′).sub.2, and Fv), so long as they exhibit the desired biological activity or properties. See, e.g. U.S. Pat. No. 4,816,567 and Mage et al., in Monoclonal Antibody Production Techniques and Applications, pp. 79-97 (Marcel Dekker, Inc.: New York, 1987).

Thus, the modifier “monoclonal” indicates the character of the antibody as being obtained from a substantially homogeneous population of antibodies, and is not to be construed as requiring a particular method. For example, the monoclonal antibodies to be used in accordance with the present invention may be made by the hybridoma method first described by Kohler and Milstein, Nature, 256:495 (1975), or may be made by recombinant DNA methods such as described in U.S. Pat. No. 4,816,567. The “monoclonal antibodies” may also be isolated from phage libraries generated using the techniques described in McCafferty et al., Nature, 348:552-554 (1990), for example.

The term “specific binding” or “specifically binds” as used herein refers to the situation in which the antibody, or antigen-binding portion thereof, will not show any significant binding (i.e., less than 10%, 9%, 8%, 7%, 6%, 5%, 4%, 3%, 2% or 1%) to molecules other than its specific binding partner(s), a peptide comprising the antigenic epitope. The term is also applicable where e.g., an antigen-binding domain of an antibody of the invention is specific for a particular epitope that is comprised by a number of antigens, in which case the specific antibody carrying the antigen-binding domain will be able to bind to the various antigens comprising the epitope. The monoclonal antibodies of the invention selectively bind to ghrelin molecules comprising SEQ ID NO: 20 and will not bind (or will bind weakly) to non-ghrlein proteins. The most preferred antibodies will specifically bind to amino acids 14-27 of human ghrelin.

The phrases “biological property” or “biological characteristic,” or the terms “biological activity” or “bioactivity,” in reference to an antibody of the present invention, are used interchangeably herein and include, but are not limited to, having the ability to modulate ghrelin activity (acylated or des-acyl), ghrelin levels or ghrelin activation, including, by way of example, change in intracellular calcium levels in at least one type of mammalian cell, in epitope/antigen affinity and specificity (e.g., anti-ghrelin monoclonal antibody binding to ghrelin), ability to antagonize an activity of the acylated or des-acyl ghrelin in vivo, in vitro, or in situ (e.g., growth hormone release), the in vivo stability of the antibody and the immunogenic properties of the antibody. Other identifiable biological properties or characteristics of an antibody recognized in the art include, for example, cross-reactivity, (i.e., with non-human homologs of the targeted peptide, or with other proteins or tissues, generally), and ability to preserve high expression levels of protein in mammalian cells. The aforementioned properties or characteristics can be observed or measured using art-recognized techniques including, but not limited to ELISA, competitive ELISA, BIAcore™ surface plasmon resonance analysis, in vitro and in vivo neutralization assays (see, e.g., Examples 2-5), and immunohistochemistry with tissue sections from different sources including human, primate, or any other source as the need may be.

The term “inhibit” or “inhibiting” means neutralizing, antagonizing, prohibiting, preventing, restraining, slowing, disrupting, stopping, or reversing progression or severity of that which is being inhibited, e.g., including, but not limited to, a biological activity or property, a disease or condition.

The term “isolated” when used in relation to a nucleic acid or protein (e.g., an antibody), refers to a nucleic acid molecule or protein molecule that is identified and separated from at least one contaminant (nucleic acid or protein, respectively) with which it is ordinarily associated in its natural source. Isolated nucleic acid or protein is present in a form or setting that is different from that in which it is found in nature. In contrast, non-isolated nucleic acids or proteins are found in the state they exist in nature. Preferably, an “isolated antibody” is an antibody that is substantially free of other antibodies having different antigenic specificities (e.g., pharmaceutical compositions of the invention comprise an isolated antibody that specifically binds ghrelin substantially free of antibodies that specifically bind antigens other than ghrelin peptide).

The terms “Kabat numbering” and “Kabat labeling” are used interchangeably herein. These terms, which are recognized in the art, refer to a system of numbering amino acid residues which are more variable (i.e., hypervariable) than other amino acid residues in the heavy and light chain variable regions of an antibody (Kabat, et al., Ann. NY Acad. Sci. 190:382-93 (1971); Kabat, et al., Sequences of Proteins of Immunological Interest, Fifth Edition, U.S. Department of Health and Human Services, NIH Publication No. 91-3242 (1991)).

A polynucleotide is “operably linked” when it is placed into a functional relationship with another polynucleotide. For example, a promoter or enhancer is operably linked to a coding sequence if it affects the transcription of the sequence.

The term “neutralizing” or “antagonizing” in reference to an anti-hGhrelin (or anti-ghrelin) monoclonal antibody of the invention or the phrase “antibody that antagonizes (neutralizes) ghrelin activity” or “antagonizes (neutralizes) ghrelin” is intended to refer to an antibody whose binding to or contact with hGhrelin results in inhibition of a biological activity induced by acylated or des-acyl human ghrelin. Inhibition of hGhrelin biological activity can be assessed by measuring one or more in vitro or in vivo indicators of hGhrelin biological activity including, but not limited to, induction of weight loss, altered feeding, or inhibition of receptor binding (see WO 01/87335 for exemplary receptor binding assay) or signal transduction in a ghrelin-receptor binding assay. Indicators of ghrelin biological activity can be assessed by one or more of the several in vitro or in vivo assays known in the art. Preferably, the ability of an anti-ghrelin antibody to neutralize or antagonize ghrelin activity is assessed by use of the FLIPR assay as described in Example 4 herein.

The terms “individual,” “subject,” and “patient,” used interchangeably herein, refer to a mammal, including, but not limited to, murines, simians, humans, mammalian farm animals, mammalian sport animals, mammalian pets and mammalian laboratory animals; preferably the term refers to humans.

The term “K_{off}” as used herein, refers to the off rate constant for dissociation of an antibody from the antibody/antigen complex. The dissociation rate constant (K_{off}) of an anti-ghrelin monoclonal antibody can be determined by BLAcore™ surface plasmon resonance as generally described in Example 5 herein. Generally, BIAcore™ analysis measures real-time binding interactions between ligand (recombinant ghrelin peptide immobilized on a biosensor matrix) and analyte (antibodies in solution) by surface plasmon resonance (SPR) using the BLAcore™ system (Pharmacia Biosensor, Piscataway, N.J.). SPR can also be performed by immobilizing the analyte (antibodies on a biosensor matrix) and presenting the ligand in solution.

The term “K_{D},” as used herein, is refers to the equilibrium dissociation constant of a particular antibody-antigen interaction. For purposes of the present invention, K_{D} is determined as shown in Example 5. Antibodies that bind a particular epitope with high affinity have a K_{D} of 10⁻⁸ M or less, more preferably 10⁻⁹ M or less and most preferably 5×10⁻¹⁰ M or less.

The term “vector” includes a nucleic acid molecule capable of transporting another nucleic acid to which it has been linked including, but not limited to, plasmids vectors, yeast expression vectors, retroviral expression vectors and other viral vectors. Certain vectors are capable of autonomous replication in a host cell into which they are introduced while other vectors can be integrated into the genome of a host cell upon introduction into the host cell, and thereby, are replicated along with the host genome. Moreover, certain vectors are capable of directing the expression of genes to which a promoter within the vector is operably linked. Such vectors are referred to herein as “recombinant expression vectors” (or simply “expression vectors”) and exemplary vectors are well known in the art.

The term “host cell” includes an individual cell or cell culture that has been a recipient of any recombinant vector(s) or isolated polynucleotide of the invention. Host cells include progeny of a single host cell, and the progeny may not necessarily be completely identical (in morphology or in total DNA complement) to the original parent cell due to natural, accidental, or deliberate mutation and/or change. A host cell includes cells transfected, transformed, electroporated or infected in vivo or in vitro with a (one or more) recombinant vector or polynucleotide of the invention. A host cell comprises a recombinant vector of the invention either stably incorporated into the host chromosome or not and may also be referred to as a “recombinant host cell”. Preferred host cells for use in the invention are CHO cells (e.g., ATCC CRL-9096), CHO-K1 cells, NS0 cells, SP2/0 cells and COS cells (ATCC e.g., CRL-1650, CRL-1651) and HeLa (ATCC CCL-2) and their derivatives and progeny. Additional host cells for use in the invention include plant cells, yeast cells and other mammalian or bacterial cells.

The present invention relates to monoclonal antibodies that specifically bind both acylated hGhrelin and des-acyl hGhrelin. Antibodies of the invention neutralize a hGhrelin or a hGhrelin biological activity whether it be acylated hGhrelin or des-acyl hGhrelin or both. The activity inhibited is preferably (i) the binding of acylated hGhrelin to receptor GHS-R1a, (ii) signal transduction prompted by acylated hGhrelin binding GHS-R1a, (iii) binding of des-acyl hGhrelin to a binding partner with which it specifically binds, or (iv) signal transduction prompted by des-acyl hGhrelin binding a binding partner with which it specifically binds. Specific binding of anti-hGhrelin monoclonal antibodies of the invention (including antigen-binding portions thereof, and humanized monoclonal antibodies with like specificity) to hGhrelin, both acylated and des-acyl forms, allows said antibodies to be used as therapeutics or prophylactics for ghrelin-associated diseases and disorders, i.e., diseases or disorders which benefit from lowering or inhibiting a ghrelin bioactivity or the level of active ghrelin present in the subject.

Epitope Identification

The epitope to which the antibodies of the invention bind is localized within amino acids 14-27 of human ghrelin (SEQ ID NO: 20). The term “epitope” refers to that portion of any molecule capable of being recognized by and bound by an antibody at one or more of the antibody's antigen-binding regions. Epitopes often consist of a chemically active surface grouping of molecules such as amino acids or sugar side chains and have specific three dimensional structural characteristics as well as specific charge characteristics. By “inhibiting epitope” and/or “neutralizing epitope” is intended an epitope, which when specifically bound by an antibody, results in loss or decrease of a biological activity of the molecule or organism containing the epitope, in vivo, in vitro or in situ.

The term “epitope,” as used herein, further refers to a portion of a polypeptide having antigenic or immunogenic activity in an animal, preferably a mammal, e.g., a mouse or a human. The term “antigenic epitope,” as used herein, is defined as a portion of a polypeptide to which an antibody can specifically bind as determined by any method well known in the art, for example, by conventional immunoassays. Antigenic epitopes need not necessarily be immunogenic, but may be immunogenic. An “immunogenic epitope,” as used herein, is defined as a portion of a polypeptide that elicits an antibody response in an animal, as determined by any method known in the art. (See, e.g., Geysen et al., Proc. Natl. Acad. Sci. USA 81:3998-4002 (1983)).

The anti-hGhrelin monoclonal antibodies of the invention (“antibodies of the invention”) specifically bind to both the acylated and des-acyl forms of hGhrelin. The epitope to which they bind, i.e., the antigenic epitope, is localized to amino acids 14-27 of human ghrelin. The antigenic epitope comprises 14, 13, 12, 11, 10, 9, 8, 7 or 6 contiguous amino acids of the peptide spanning amino acid residues 14-27 (inclusive) of human ghrelin (see SEQ ID NO: 19). Said antigenic epitope may possess additional ghrelin residues outside of amino acids 14-27 of human ghrelin, but the monoclonal antibodies of the invention do not require these additional residues to specifically bind the antigenic epitope. Additional residues of hGhrelin outside of the amino acids 14-27 antigenic epitope may affect the conformational structure of the antigenic domain and thereby alter binding properties of an antibody of the invention to the antigenic epitope. However, monoclonal antibodies of the invention specifically bind to full-length human ghrelin regardless of whether or not it is acylated. The monoclonal antibodies of the invention bind acylated hGhrelin with no greater than six-fold or five-fold; more preferably no greater than four-fold or three-fold, and most preferably no greater than two fold difference than with which it binds des-acyl hGhrelin as determined e.g., by ELISA or K_{D} values in a Biacore™ assay.

ELISA, BLAcore™ and FLIPR assays as described in the Examples section herein demonstrate that Fabs 3281, 4731 and 4281 bind a similar epitope localized within amino acids 14-27 of human or rat acylated or des-acyl ghrelin, indicating that the acyl group at amino acid three of hGhrelin is not a part of the epitope. Rat ghrelin is identical to human ghrelin except at amino acids 11 and 12. These data indicate that amino acids 11 and 12 are not a part of the epitope to which Fabs of the invention bind. Furthermore, hGhrelin 20-28 and hGhrelin 18-28 do not compete with full-length hGhrelin for binding Fabs of the invention. There is no statistical competition seen with the hGhrelin 20-28 or 18-28 peptide with full-length hGhrelin for binding Fab 3281. These data indicate that ghrelin polypeptides spanning amino acids 20-28 or amino acids 18-28 do not provide the complete epitope. However, ghrelin polypeptides spanning amino acids 1-28 or 1-27 or 14-28 of human ghrelin do provide the complete antigenic epitope. It is commonly believed in the art that a linear epitope has an optimal length of 8-12 amino acids and that the minimal size of a linear epitope is about 6 amino acid residues. However, a linear epitope may be greater than 30 amino acids in length (See e.g., Oleksiewicz, M B et al., J. Virology, 75:3277-3290, 2001; Torrez-Martinez, N., et al., Virology, 211: 336-338, 1995).

The domain spanning amino acids 14-27 (inclusive) of hGhrelin may also be used as an immunogenic antigen to generate monoclonal antibodies of the invention. This domain (i.e., QRKESKKPPAKLWP, SEQ ID NO:20) or a fusion protein thereof, may be used to immunize a non-human animal, preferably a mouse. Various methods for the preparation of antibodies are well known in the art. For example, antibodies may be prepared by immunizing a suitable mammalian host using a ghrelin protein, peptide, or fragment, in isolated or immunoconjugated form (Harlow, Antibodies, Cold Spring Harbor Press, NY (1989). In addition, fusion protein of ghrelin or the antigenic peptide may also be used. Cells expression or overexpressing ghrelin may also be used for immunizations. Similarly, any cell engineered to express ghrelin or an antigenic peptide of ghrelin may be used.

In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-1031). Immortalized cell lines are usually transformed mammalian cells, particularly myeloma cells of rodent, bovine and human origin. Usually, rat or mouse myeloma cell lines are employed using the standard method of Kohler and Milstein or modifications as generally known. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. An example of such a murine myeloma cell line is P3X63AgU.1. Human myeloma and mouse-human heteromyeloma cell lines also have been described for the production of human monoclonal antibodies [Kozbor, J. Immunol., 133:3001 (1984); Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against the antigenic epitope or a peptide comprising the antigenic epitope. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are well-known in the art. The binding affinity of the monoclonal antibody can, for example, be determined by the Scatchard analysis of Munson and Pollard, Anal. Biochem., 107:220 (1980) or by BIAcore™ assay.

When the appropriate immortalized cell culture secreting the desired antibody is identified, the cells can be cultured either in vitro or by production in ascites fluid. After the desired hybridoma cells are identified, the clones may be subcloned by limiting dilution procedures and grown by standard methods [Goding, supra). Suitable culture media for this purpose include, for example, Dulbecco's Modified Eagle's Medium or RPMI-1640 medium. Alternatively, the hybridoma cells may be grown in vivo as ascites in a mammal. The monoclonal antibodies secreted by the subclones may be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies may also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA may be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences (U.S. Pat. No. 4,816,567) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Anti-hGhrelin antibodies are isolated from the immunized animal and screened by methods well known in the art to isolate those antibodies that specifically bind a peptide spanning amino acids 14-27 of both the acylated and des-acyl forms of hGhrelin. Methods for such isolation and screening are well known in the art. [See, e.g., Kohler and Milstein, Nature, 256:495 (1975), Goding, Monoclonal Antibodies: Principles and Practice, Academic Press, (1986) pp. 59-1031, Kozbor, J. Immunol., 133:3001 (1984); Brodeur et al., Monoclonal Antibody Production Techniques and Applications, Marcel Dekker, Inc., New York, (1987) pp. 51-63), U.S. Pat. No. 4,816,567].

The antibodies of the invention may also comprise monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in U.S. Pat. No. 4,342,566. Papain digestion of antibodies typically produces two identical antigen binding fragments, called Fab fragments, each with a single antigen binding site, and a residual Fc fragment. Pepsin treatment yields an F(ab′)₂ fragment that has two antigen combining sites and is still capable of cross-linking antigen.

The Fab fragments produced in the antibody digestion also contain the constant domains of the light chain and the first constant domain of the heavy chain. Fab′ fragments differ from Fab fragments by the addition of a few residues at the carboxy terminus of the heavy chain CH, domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known. Single chain Fv fragments may also be produced, such as described in Iliades et al., FEBS Letters, 409:437-441, 1997. Coupling of such single chain fragments using various linkers is described in Kortt et al., Protein Engineering, 10:423-433 (1997). Isolated antibodies may further be altered to a chimeric or humanized form using methods well known in the art. Monoclonal anti-hGhrelin antibodies isolated by this process are contemplated to fall within the scope of the invention.

In a preferred embodiment, the invention provides isolated anti-hGhrelin monoclonal antibodies that preferably bind a human ghrelin peptide comprising or consisting of the epitope located within amino acids 14-27 of human ghrelin (acylated or des-acyl) with an equilibrium dissociation constant, K_{D}, of 10⁻⁷ or 10⁻⁸ M or less and more preferably 10⁻⁹ M or less (as determined by solid phase BIAcore™ surface plasmon resonance at room temperature) and has the capacity to antagonize an activity of human ghrelin.

Anti-hGhrelin monoclonal antibodies of the invention inhibit a hGhrelin-mediated activity as represented, e.g., by the FLIPR assay described in Examples 3 and 4 herein. Preferably, said hGhrelin-mediated activity is inhibited with an IC₅₀ of 40 nM or less, more preferably 20 nM or less, 10 nM or less, 5 nM or less, 4 nM or less, 3 nM or less, most preferably 2 nM or less, or 1 nM or less or an IC₅₀ of 0.8 nM or less.

In one embodiment, preferred anti-hGhrelin Fab are those referred to herein as 3281 and 4731. The 3281 Fab has a LCVR and a HCVR comprising a peptide with a sequence as shown in SEQ ID NO: 13 and SEQ ID NO: 15 respectively. The 4731 Fab has a LCVR and a HCVR comprising a peptide with a sequence as shown in SEQ ID NO: 14 and SEQ ID NO: 15 respectively. Exemplary polynucleotide sequences encoding the LCVR and HCVR of Fab 4731 are shown in SEQ ID NO: 17 and SEQ ID NO: 18 respectively.

The present invention is also directed to cell lines that produce an anti-hGhrelin monoclonal antibody of the invention or an antigen-binding fragment thereof. Creation and isolation of cell lines producing a monoclonal antibody of the invention can be accomplished using routine techniques known in the art. Preferred cell lines include COS, CHO, SP2/0, NS0, HeLa and yeast (available from public repositories such as ATCC, American Type Culture Collection, Manassas, Va.).

A wide variety of host expression systems can be used to express an antibody of the present invention including prokaryotic and eukaryotic expression systems (such as yeast, baculoviral, plant, mammalian and other animal cells, transgenic animals, and hybridoma cells), as well as phage display expression systems. An example of a suitable bacterial expression vector is pUC119 and a suitable eukaryotic expression vector is a modified pcDNA3.1 vector with a weakened DHFR selection system. Other antibody expression systems are also known in the art and are contemplated herein.

An antibody of the invention can be prepared by recombinant expression of immunoglobulin light and heavy chain genes in a host cell. To express an antibody recombinantly, one or more recombinant expression vectors carrying DNA fragments encoding the immunoglobulin light and/or heavy chains of an antibody of the invention are introduced into a host cell via transfection, transformation, infection, or the like, such that the antibody of the invention, or antigen-binding fragment thereof, are expressed in the host cell. Preferably, the antibody is secreted into the medium in which the host cells are cultured, from it can be recovered or purified. Standard recombinant DNA methodologies are used to obtain antibody heavy and light chain genes, incorporate these genes into recombinant expression vectors, and introduce the vectors into host cells. Such standard recombinant DNA technologies are described, for example, in Sambrook, Fritsch, and Maniatis (Eds.), Molecular Cloning; A Laboratory Manual, Second Edition, Cold Spring Harbor, N.Y., (1989); and Ausubel, et al (Eds.) Current Protocols in Molecular Biology, Greene Publishing Associates, (1989).

An isolated DNA encoding a HCVR region can be converted to a full-length heavy chain gene by operably linking the HCVR-encoding DNA to another DNA molecule encoding heavy chain constant regions (CH1, CH2, and CH3). The sequences of human heavy chain constant region genes are known in the art. See, e.g., Kabat, et al., Sequences of Proteins of Immunological Interest, Fifth Edition, U.S. Department of Health and Human Services, NIH Publication No. 91-3242 (1991). DNA fragments encompassing these regions can be obtained by standard PCR amplification. The heavy chain constant region can be of any type, (e.g., IgG, IgA, IgE, IgM or IgD), class (e.g., IgG₁, IgG₂, IgG₃ and IgG₄) or subclass constant region and any allotypic variant thereof as described in Kabat (supra), but most preferably is an IgG₄ or an IgG₁ constant region. Alternatively, the antigen binding portion can be a Fab fragment, Fab′ fragment, F(ab′)₂ fragment, Fd, or a single chain Fv fragment (scFv). For a Fab fragment heavy chain gene, the HCVR-encoding DNA can be operably linked to another DNA molecule encoding only a heavy chain CH1 constant region.

An isolated DNA encoding a LCVR region can be converted to a full-length light chain gene (as well as a Fab light chain gene) by operably linking the LCVR-encoding DNA to another DNA molecule encoding a light chain constant region, CL. The sequences of human light chain constant region genes are known in the art. See, e.g., Kabat, supra. DNA fragments encompassing these regions can be obtained by standard PCR amplification. The light chain constant region can be a kappa or lambda constant region.

To create an scFv gene, the HCVR- and LCVR-encoding DNA fragments are operably linked to another fragment encoding a flexible linker, e.g., encoding the amino acid sequence (Gly₄-Ser)₃, such that the HCVR and LCVR sequences can be expressed as a contiguous single-chain protein, with the LCVR and HCVR regions joined by the flexible linker. See, e.g., Bird, et al., Science 242:423-426 (1988); Huston, et al., Proc. Natl. Acad. Sci. USA 85:5879-5883 (1988); McCafferty, et al., Nature 348:552-554 (s990).

To express an antibody of the invention, a DNA comprising a partial or full-length light and/or heavy chain, obtained as described above, is inserted into an expression vector such that the gene is operably linked to transcriptional and translational control sequences. The partial or full-length light and heavy chains may each be operably linked to a separate promoter sequence or they may be operably linked to one promoter. If the sequences comprising LCVR and HCVR (said sequence may further be operably linked to the constant region of the antibody) are present in the same vector and transcribed from one promoter to which they are both operably linked, a sequence comprising LCVR may be 5′ or 3′ to a sequence comprising HCVR. Furthermore, the LCVR and HCVR coding region in the vector may be separated by a linker sequence of any size or content, preferably such linker, when present, comprises a sequence encoding an internal ribosome entry site.

The expression vector and expression control sequences are chosen to be compatible with the expression host cell used. The antibody light chain gene and the antibody heavy chain gene can be inserted into separate expression vectors or, more typically, both genes are inserted into the same expression vector. The antibody genes are inserted into the expression vector by standard methods. Additionally, the recombinant expression vector can encode a signal peptide that facilitates secretion of the anti-ghrelin monoclonal antibody light and/or heavy chain from a host cell. The anti-ghrelin monoclonal antibody light and/or heavy chain gene can be cloned into the vector such that the signal peptide is operably linked in-frame to the amino terminus of the antibody chain gene. The signal peptide can be an immunoglobulin signal peptide or a heterologous signal peptide.

In addition to the antibody heavy and/or light chain gene(s), a recombinant expression vector of the invention carries regulatory sequences that control the expression of the antibody chain gene(s) in a host cell. The term “regulatory sequence” is intended to include promoters, enhancers and other expression control elements (e.g., polyadenylation signals), as needed, that control the transcription or translation of the antibody chain gene(s). The design of the expression vector, including the selection of regulatory sequences may depend on such factors as the choice of the host cell to be transformed, the level of expression of protein desired. Preferred regulatory sequences for mammalian host cell expression include viral elements that direct high levels of protein expression in mammalian cells, such as promoters and/or enhancers derived from cytomegalovirus (CMV), Simian Virus 40 (SV40), adenovirus, (e.g., the adenovirus major late promoter (AdMLP)) and polyoma virus.

In addition to the antibody heavy and/or light chain genes and regulatory sequences, the recombinant expression vectors of the invention may carry additional sequences, such as sequences that regulate replication of the vector in host cells (e.g., origins of replication) and one or more selectable marker genes. The selectable marker gene facilitates selection of host cells into which the vector has been introduced. For example, typically the selectable marker gene confers resistance to drugs, such as G418, hygromycin, or methotrexate, on a host cell into which the vector has been introduced. Preferred selectable marker genes include the dihydrofolate reductase (DHFR) gene (for use in DHFR-minus host cells with methotrexate selection/amplification), the neo gene (for G418 selection), and glutanine synthetase (GS) in a GS-negative cell line (such as NS0) for selection/amplification.

For expression of the light and/or heavy chains, the expression vector(s) encoding the heavy and/or light chains is transfected into a host cell by standard techniques e.g., electroporation, calcium phosphate precipitation, DEAE-dextran transfection and the like. Although it is theoretically possible to express the antibodies of the invention in either prokaryotic or eukaryotic host cells, preferably eukaryotic cells, and most preferably mammalian host cells, because such cells, are more likely to assemble and secrete a properly folded and immunologically active antibody. Preferred mammalian host cells for expressing the recombinant antibodies of the invention include Chinese Hamster Ovary (CHO cells) (including DHFR-CHO cells, described in Urlaub and Chasin, Proc. Natl. Acad. Sci. USA 77:4216-20 (1980), used with a DHFR selectable marker, e.g., as described in Kaufman and Sharp, J. Mol. Biol. 159:601-21 (1982)), NS0 myeloma cells, COS cells, HeLa cells and SP2/0 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the host cell and/or the culture medium using standard purification methods.

Host cells can also be used to produce portions, or fragments, of intact antibodies, e.g., Fab fragments or scFv molecules. It will be understood that variations on the above procedure are within the scope of the present invention. For example, it may be desirable to transfect, transform, electorporate, or the like, a host cell with DNA encoding either the light chain or the heavy chain (but not both) of an antibody of this invention. Recombinant DNA technology may also be used to remove some or all the DNA encoding either or both of the light and heavy chains that is not necessary for binding to ghrelin. The molecules expressed from such truncated DNA molecules are also encompassed by the antibodies of the invention.

In a preferred system for recombinant expression of an antibody of the invention, a recombinant expression vector encoding both the antibody heavy chain and the antibody light chain is introduced into DHFR-CHO cells by calcium phosphate-mediated transfection. Within the recombinant expression vector, the antibody heavy and light chain genes are each operably linked to separate enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells and recover the antibody from the culture medium. Antibodies, or antigen-binding portions thereof, of the invention can be expressed in an animal (e.g., a mouse) that is transgenic for human immunoglobulin genes (see, e.g., Taylor, et al., Nucleic Acids Res. 20:6287-95(1992)). Plant cells can also be modified to create transgenic plants that express the antibody, or an antigen-binding portion thereof, of the invention.

The invention also provides recombinant expression vectors encoding both an antibody heavy chain and/or an antibody light chain. For example, in one embodiment, the invention provides a recombinant expression vector encoding:
a) an antibody heavy chain having a variable region comprising at least one peptide with an amino acid sequence selected from the group consisting of SEQ ID NOs: 6-12; and further comprising sequence encoding
b) an antibody light chain having a variable region comprising at least one peptide with an amino acid sequence selected from the group consisting of SEQ ID NOs: 1-5.

The invention also provides host cells into which one or more of the recombinant expression vectors of the invention have been introduced. Preferably, the host cell is a mammalian host cell, more preferably the host cell is a CHO cell, an NS0 cell, a SP2/0 cell, a COS cell. Such cells are available from biological repositories such as the ATCC in Manassas, Va. Still further the invention provides a method of synthesizing an antibody of the invention by culturing a host cell of the invention in a suitable culture medium until said antibody of the invention is synthesized. The method can further comprise isolating the antibody from the culture medium.

Once expressed, the intact antibodies, their dimers, individual light and heavy chains, or other immunoglobulin forms of the present invention can be purified according to standard procedures of the art, including ammonium sulfate precipitation, ion exchange, affinity, reverse phase, hydrophobic interaction column chromatography, gel electrophoresis and the like. Substantially pure immunoglobulins of at least about 90%, 92%, 94% or 96% homogeneity are preferred, and 98 to 99% or more homogeneity most preferred, for pharmaceutical uses. Once purified, partially or to homogeneity as desired, the peptides may then be used therapeutically or prophylactically, as directed herein.

Chimeric Antibodies

As used herein, the term “chimeric antibody” includes monovalent, divalent or polyvalent immunoglobulins. A monovalent chimeric antibody is a dimer formed by a chimeric heavy chain associated through disulfide bridges with a chimeric light chain. A divalent chimeric antibody is a tetramer formed by two heavy chain-light chain dimers associated through at least one disulfide bridge.

A chimeric heavy chain comprises an antigen-binding region derived from the heavy chain of a non-human antibody specific for ghrelin, which is linked to at least a portion of a human heavy chain constant region, such as CH1 or CH2. A chimeric light chain comprises an antigen binding region derived from the light chain of a non-human antibody specific for ghrelin, linked to at least a portion of a human light chain constant region (CL).

Antibodies, fragments or derivatives having chimeric heavy chains and light chains of the same or different variable region binding specificity, can also be prepared by appropriate association of the individual polypeptide chains, according to known method steps. With this approach, hosts expressing chimeric heavy chains are separately cultured from hosts expressing chimeric light chains, and the immunoglobulin chains are separately recovered and then associated. Alternatively, the hosts can be co-cultured and the chains allowed to associate spontaneously in the culture medium, followed by recovery of the assembled immunoglobulin or fragment.

Methods for producing chimeric antibodies are known in the art (see, e.g., U.S. Pat. Nos. 6,284,471; 5,807,715; 4,816,567; and 4,816,397).

In a preferred embodiment, a gene is created which comprises a first DNA segment that encodes at least the antigen-binding region of non-human origin (e.g., that of Fab 1181 or Fab 1621), such as functionally rearranged variable (V) region with joining (J) segment, linked to a second DNA segment encoding at least a part of a human constant (C) region as described in U.S. Pat. No. 6,284,471 (incorporated herein in its entirety).

Humanized Antibodies

A “humanized antibody” has CDRs that originate from a non-human (preferably a mouse monoclonal antibody) while framework and constant region, to the extent it is present, (or a substantial portion thereof, i.e., at least about 90%, 92%, 94%, 96%, 98% or 99%) are encoded by nucleic acid sequence information that occurs in the human germline immunoglobulin region or in recombined or mutated forms thereof whether or not said antibodies are produced in human cells. A humanized antibody may be an intact antibody, a substantially intact antibody, a portion of an antibody comprising an antigen-binding site, or a portion of an antibody comprising a Fab fragment, Fab′ fragment, F(ab′)₂, or a single chain Fv fragment. It is contemplated that in the process of creating a humanized antibody, the amino acid at either termini of a CDR (see e.g., SEQ ID NOs:1-12) may be substituted with an amino acid that occurs in the human gemline for that segment of adjoining framework sequence. Preferably a therapeutic antibody of the invention would have sequence of the framework and/or constant region derived from the mammal in which it would be used as a therapeutic so as to decrease the possibility that the mammal would illicit an immune response against the therapeutic antibody.

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522-5251986); Riechmann et al., Nature, 332:323-327 (1988); Verhoeyen et al., Science, 239:1534-1536, 1988), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some framework residues are substituted by residues from analogous sites in rodent antibodies.

The choice of human variable domains, both light and heavy, to be used in making the humanized antibodies aid in reducing antigenicity. According to the “best-fit” method, the sequence of the variable domain of a rodent antibody is screened against the entire library of known human variable domain sequences. The human sequence which is closest to that of the rodent is then accepted as the human framework (FR) for the humanized antibody (Sims et al., J. Immunol., 151:2296-2308, 1993; Chothia and Lesk, J. Mol. Biol., 196:901-917, 1987). Another method uses a particular framework derived from the consensus sequence of all human antibodies of a particular subgroup of light or heavy chains. The same framework may be used for several different humanized antibodies (Carter et al., Proc. Natl. Acad. Sci. USA, 89:4285-4289, 1992; Presta et al., J. Immunol., 151:2623-2632, 1993). Any art-known means for selecting human framework for use in the humanized antibody may be used with the present invention.

Humanized antibodies may be subjected to in vitro mutagenesis using methods of use in the art (or, when an animal transgenic for human Ig sequences is used, in vivo somatic mutagenesis) and, thus, the framework region amino acid sequences of the HCVR and LCVR regions of the humanized recombinant antibodies are sequences that, while derived from those related to human germline HCVR and LCVR sequences, may not naturally exist within the human antibody germline repertoire in vivo. It is contemplated that such amino acid sequences of the HCVR and LCVR framework regions of the humanized recombinant antibodies are at least 90%, 92%, 94%, 95%, 96%, 97%, 98% or most preferably at least 99% identical to a human germline sequence.

Humanized antibodies have at least three potential advantages over non-human and chimeric antibodies for use in human therapy: (i) the effector portion is human, it may interact better with the other parts of the human immune system (e.g., destroy the target cells more efficiently by complement-dependent cytotoxicity or antibody-dependent cellular cytotoxicity); (ii) the human immune system should not recognize the framework or constant region of the humanized antibody as foreign, and therefore the antibody response against such an injected antibody should be less than that against a totally foreign non-human antibody or a partially foreign chimeric antibody; and (iii) injected non-human antibodies have been reported to have a half-life in the human circulation much shorter than the half-life of human antibodies. Injected humanized antibodies may have a half-life much like that of naturally occurring human antibodies, thereby allowing smaller and less frequent doses to be given.

Humanization may in some instances adversely affect antigen binding of the antibody. Preferably a humanized anti-hGhrelin monoclonal antibody of the present invention will possess a binding affinity for hGhrelin of not less than about 50%, more preferably not less than about 30%, and most preferably not less than about 5% of the binding affinity of the parent murine antibody, preferably Fab 3281, 4731 or Fab 4281, for hGhrelin. Preferably, a humanized antibody of the present invention will bind the same epitope as does Fab 3281, 4731 or 4281 described herein. Said antibody can be identified based on its ability to compete with Fab 3281, 4731 or 4281 for binding to acylated hGhrelin or des-acyl hGhrelin or to cells expressing acylated hGhrelin or des-acyl hGhrelin.

The design of humanized antibodies of the invention may be carried out as follows. In general, the humanized antibodies are produced by obtaining nucleic acid sequences encoding the HCVR and LCVR of an antibody which binds a hGhrelin epitope localized between amino acids 14-27 of hGhrelin, identifying the CDRs in said HCVR and LCVR (nonhuman), and grafting such CDR-encoding nucleic acid sequences onto selected human framework-encoding nucleic acid sequences. Preferably, the human framework amino acid sequences are selected such that the resulting antibody is likely to be suitable for in vivo administration in humans. This can be determined, e.g., based on previous usage of antibodies containing such human framework sequence. Preferably, the human framework sequence will not itself be significantly immunogenic.

Alternatively, the amino acid sequences of the frameworks for the antibody to be humanized (e.g., Fab 3281) will be compared to those of known human framework sequences the human framework sequences to be used for CDR-grafting will be selected based on their comprising sequences highly similar to those of the parent antibody, e.g., a murine antibody which binds hGhrelin. Numerous human framework sequences have been isolated and their sequences reported in the art. This enhances the likelihood that the resultant CDR-grafted humanized antibody, which contains CDRs of the parent (e.g., murine) antibody grafted onto selected human frameworks (and possibly also the human constant region) will substantially retain the antigen binding structure and thus retain the binding affinity of the parent antibody. To retain a significant degree of antigen binding affinity, the selected human framework regions will preferably be those that are expected to be suitable for in vivo administration, i.e., not immunogenic.

In either method, the DNA sequence encoding the HCVR and LCVR regions of the preferably murine anti-hGhrelin antibody are obtained. Methods for cloning nucleic acid sequences encoding immunoglobulins are well known in the art. Such methods may, for example, involve the amplification of the immunoglobulin-encoding sequences to be cloned using appropriate primers by polymerase chain reaction (PCR). Primers suitable for amplifying immunoglobulin nucleic acid sequences, and specifically murine HCVR and LCVR sequences have been reported in the literature. After such immunoglobulin-encoding sequences have been cloned, they will be sequences by methods well known in the art.

Once the DNA sequences encoding the CDRs and frameworks of the antibody which is to be humanized have been identified, (see e.g., Tables 1 and 2 herein), the amino acid sequences encoding the CDRs are then identified (deduced based on the nucleic acid sequences and the genetic code and by comparison to previous antibody sequences) and the CDR-encoding nucleic acid sequences are grafted onto selected human framework-encoding sequences. This may be accomplished by use of appropriate primers and linkers. Methods for selecting suitable primers and linkers for ligation of desired nucleic acid sequences is well within the ability of one of ordinary skill in the art.

After the CDR-encoding sequences are grafted onto the selected human framework encoding sequences, the resultant DNA sequences encoding the “humanized” variable heavy and variable light sequences are then expressed to produce a humanized Fv or humanized antibody which specifically binds acylated and des-acyl hGhrelin at the antigenic peptide of the invention (i.e., amino acids 14-27 of human ghrelin). Typically, the humanized HCVR and LCVR are expressed as part of a whole anti-hGhrelin antibody molecule, i.e., as a fusion protein with human constant domain sequences whose encoding DNA sequences have been obtained from a commercially available library or which have been obtained using, e.g., one of the above described methods for obtaining DNA sequences, or are in the art. However, the HCVR and LCVR sequences can also be expressed in the absence of constant sequences to produce a humanized anti-hGhrelin Fv. Nevertheless, fusion of human constant sequences is potentially desirable because the resultant humanized anti-hGhrelin antibody may possess human effector functions.

Methods for synthesizing DNA encoding a protein of known sequence are well known in the art. Using such methods, DNA sequences which encode the subject humanized HCVR and LCVR sequences (with or without constant regions) are synthesized, and then expressed in a vector system suitable for expression of recombinant antibodies. This may be effected in any vector system which provides for the subject humanized HCVR and LCVR sequences to be expressed as a fusion protein with human constant domain sequences and to associate to produce functional (antigen binding) antibodies or antibody fragments.

Human constant domain sequences are well known in the art and have been reported in the literature. Preferred human constant light chain sequences include the kappa and lambda constant light chain sequences. Preferred human constant heavy chain sequences include human gamma 1, human gamma 2, human gamma 3, human gamma r, and mutated versions thereof which provide for altered effect or function, e.g., enhanced in vivo half-life, reduced Fc receptor binding, and the like.

In some instances, humanized antibodies produced by grafting CDRs (from an antibody of the invention which binds the antigenic peptide of the invention, 14-27 of hGhrelin) onto selected human frameworks may provide humanized antibodies having the desired affinity to hGhrelin. However, it may be necessary or desirable to further modify specific residues of the selected human framework in order to enhance antigen binding. Preferably, those framework residues of the parent (e.g., murine) antibody which maintain or affect combining-site structures will be retained. These residues may be identified by X-ray crystallography of the parent antibody or Fab fragment, thereby identifying the three-dimensional structure of the antigen-binding site.

References further describing methods involved in humanizing a mouse antibody that may be used are e.g., Queen et al., Proc. Natl. Acad. Sci. USA 88:2869, 1991; U.S. Pat. No. 5,693,761; U.S. Pat. No. 4,816,397; U.S. Pat. No. 5,225,539; computer programs ABMOD and ENCAD as described in Levitt, M., J. Mol. Biol. 168:595-620, 1983.

The present invention further embraces variants and the equivalents that are substantially homologous to the humanized antibodies and antibody fragments set forth herein. These are contemplated to contain 1 or 2 conservative substitution mutations within the CDRs of the antibody. Conservative amino acid substitutions can frequently be made in a protein without altering either the conformation or the function of the protein. Conservative substitution refers to the substitution of an amino acid with another within the same general class, e.g., one acidic amino acid with another acidic amino acid, one basic amino acid with another basic amino acid, one hydrophobic amino acid with another hydrophobic amino acid or one neutral amino acid by another neutral amino acid. Other substitutions can also be considered conservative, depending on the environment of the particular amino acid and its role in the three-dimensional structure of the protein. For example, glycine and alanine can frequently be interchangeable as can alanaine and valine. What is intended by a conservative amino acid substitution is well known in the art. These variants and equivalents substantially homologous to the humanized antibodies are also contemplated to contain a deletion of a terminal amino acid of a CDR.

Diagnostic Use

An antibody of the invention may be used to diagnose a disorder or disease associated with the expression of human ghrelin, i.e., either acylated or des-acyl form of ghrelin. In a similar manner, the antibody of the invention can be used in an assay to monitor ghrelin levels in a subject being treated or being considered for treatment for a ghrelin-associated condition (e.g., obesity, obesity-related disorders, NIDDM (Type II diabetes), Prader-Willi syndrome, eating disorders, hyperphagia, impaired satiety, anxiety, gastric motility disorders (including e.g., irritable bowel syndrome and functional dyspepsia), insulin resistance syndrome, metabolic syndrome, dyslipidemia, atherosclerosis, hypertension, hyperandrogenism, polycystic ovarian syndrome, cancer, and cardiovascular disorders). Diagnostic assays include methods that utilize the antibody of the invention and a label to detect acylated ghrelin and/or des-acyl ghrelin in a sample, e.g., in a human body fluid or in a cell or tissue extract. Binding compositions, such as, e.g., antibodies, are used with or without modification, and are labeled by covalent or non-covalent attachment of a reporter molecule.

A variety of conventional protocols for measuring ghrelin, including ELISAs, RIAs, and FACS, are known in the art and provide a basis for diagnosing altered or abnormal levels of ghrelin expression. Normal or standard expression values are established using any art known technique, e.g., by combining a sample comprising a ghrelin polypeptide with, e.g., antibodies under conditions suitable to form a ghrelin:antibody complex. The antibody is directly or indirectly labeled with a detectable substance to facilitate detection of the bound or unbound antibody. Suitable detectable substances include various enzymes, prosthetic groups, fluorescent materials, luminescent materials and radioactive materials. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of a radioactive material include ¹²⁵I, ¹³¹I, ³⁵S, or ³H. (See, e.g., Zola, Monoclonal Antibodies: A Manual of Techniques, CRC Press, Inc. (1987)).

The amount of a standard complex formed is quantitated by various methods, such as, e.g., photometric means. Amounts of ghrelin polypeptide expressed in subject, control, and samples (e.g., from biopsied tissue) are then compared with the standard values. Deviation between standard and subject values establishes parameters for correlating a particular disorder, state, condition, syndrome, or disease with a certain level of expression (or lack thereof) for a ghrelin polypeptide.

Once the presence of a disorder, state, condition, syndrome, or disease is established and a treatment protocol is initiated, assays are repeated on a regular basis to monitor the level of ghrelin expression. The results obtained from successive assays are used to show the efficacy of treatment over a period ranging from several days to months. With respect to disorders of cell proliferation (e.g., a cancer), the presence of an abnormal amount of ghrelin (either under- or over expressed) in biopsied tissue or fluid from a subject may indicate a predisposition for the development of a disorder, state, condition, syndrome, or disease of cell proliferation or it may provide a means for detecting such a disorder, state, condition, syndrome, or disease prior to the appearance of actual clinical symptoms. A more definitive initial detection may allow earlier treatment thereby preventing and/or ameliorating further progression of cell proliferation.

Pharmaceutical Composition

An antibody of the invention can be incorporated into pharmaceutical compositions suitable for administration to a subject. The compounds of the invention may be administered alone or in combination with a pharmaceutically acceptable carrier, diluent, and/or excipients, in single or multiple doses. The pharmaceutical compositions for administration are designed to be appropriate for the selected mode of administration, and pharmaceutically acceptable diluents, carrier, and/or excipients such as dispersing agents, buffers, surfactants, preservatives, solubilizing agents, isotonicity agents, stabilizing agents and the like are used as appropriate. Said compositions are designed in accordance with conventional techniques as in e.g., Remington, The Science and Practice of Pharmacy, 19^{th} Edition, Gennaro, Ed., Mack Publishing Co., Easton, Pa. 1995 which provides a compendium of formulation techniques as are generally known to practitioners. Suitable carriers for pharmaceutical compositions include any material which when combined with a monoclonal antibody of the invention retains the molecule's activity and is non-reactive with the subject's immune system.

A pharmaceutical composition comprising an anti-hGhrelin monoclonal antibody of the present invention can be administered to a subject at risk for or exhibiting pathologies associated with obesity or related disorders as described herein using standard administration techniques including oral, intravenous, intraperitoneal, subcutaneous, pulmonary, transdermal, intramuscular, intranasal, buccal, sublingual, or suppository administration.

A pharmaceutical composition of the invention preferably is a “therapeutically effective amount” or a “prophylactically effective amount” of an antibody of the invention. A “therapeutically effective amount” refers to an amount effective, at dosages and for periods of time necessary, to achieve the desired therapeutic result. A therapeutically effective amount of the antibody may vary according to factors such as the disease state, age, sex, and weight of the individual, and the ability of the antibody or antibody portion to elicit a desired response in the individual. A therapeutically effective amount is also one in which any toxic or detrimental effect of the antibody, are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

A therapeutically-effective amount is at least the minimal dose, but less than a toxic dose, of an active agent which is necessary to impart therapeutic benefit to a subject. Stated another way, a therapeutically-effective amount for treating obesity is an amount which induces, ameliorates or otherwise causes an improvement in the obese state of the mammal, e.g., by decreasing body mass index (BMI).

The route of administration of an antibody of the present invention may be oral, parenteral, by inhalation, or topical. Preferably, the antibodies of the invention can be incorporated into a pharmaceutical composition suitable for parenteral administration. The term parenteral as used herein includes intravenous, intramuscular, subcutaneous, rectal, vaginal, or intraperitoneal administration. Peripheral systemic delivery by intravenous or intraperitoneal or subcutaneous injection is preferred. Suitable vehicles for such injections are straightforward.

The pharmaceutical composition typically must be sterile and stable under the conditions of manufacture and storage in the container provided, including e.g., a sealed vial or syringe. Therefore, pharmaceutical compositions may be sterile filtered after making the formulation, or otherwise made microbiologically acceptable. A typical composition for intravenous infusion could have a volume as much as 250-1000 ml of fluid, such as sterile Ringer's solution, physiological saline, dextrose solution and Hank's solution and a therapeutically effective dose, (e.g., 1 to 100 mg/mL, or more) of antibody concentration. Therapeutic agents of the invention may be frozen or lyophilized for storage and reconstituted in a suitable sterile carrier prior to use. Lyophilization and reconstitution can lead to varying degrees of antibody activity loss (e.g., with conventional immunoglobulins, IgM antibodies tend to have greater activity loss than IgG antibodies). Dosages may have to be adjusted to compensate. Generally, pH between 6 and 8 is preferred.

As is well known in the medical arts, dosages for any one subject depends upon many factors, including the patient's size, body surface area, age, the particular compound to be administered, sex, time and route of administration, general health, and other drugs being administered concurrently. A typical dose can be, for example, in the range of 0.001 to 1000 μg; however, doses below or above this exemplary range are envisioned, especially considering the aforementioned factors. The daily parenteral dosage regimen is about 0.1 μg/kg to about 100 mg/kg of total body weight, preferably from about 0.3 μg/kg to about 10 mg/kg and more preferably from about 1 μg/kg to 1 mg/kg, even more preferably from about 0.5 to 10 mg/kg body weight per day. Progress may be monitored by periodic assessment.

Therapeutic Use

Ghrelin plays a role in the pathophysiology of obesity and a number of related disorders or diseases. Ghrelin is the first circulating hormone shown to stimulate feeding in humans following systemic administration. One study demonstrated that obese subjects do not demonstrate the decline in plasma ghrelin levels as seen after a meal in lean subjects and may therefore lead to increased food consumption (English, P. et al, J. Clin. End. & Metabolism, 87:2984-2987, 2002). Therefore, a pharmaceutical composition comprising an anti-hGhrelin monoclonal antibody of the invention may be used to treat or prevent obesity and/or obesity-related disorders such as NIDDM, Prader-Willi syndrome, impaired satiety, hyperphagia.

Obesity, also called corpulence or fatness, is the excessive accumulation of body fat, usually caused by the consumption of more calories than the body uses. The excess calories are then stored as fat, or adipose tissue. To be overweight, if moderate, is not necessarily to be obese, e.g., in muscular individuals. In general, however, a body weight of a subject that is 20 percent or more over the optimum tends to be associated with obesity. Alternatively, obesity may be defined in terms of Body Mass Index (BMI). Human BMI is defined as the body weight of a human in kilograms divided by the square of the height of that individual in meters. Typically, persons with a BMI of between 25 and 29 are considered overweight and a BMI of 29 or greater is considered obese. This may vary in some persons due to differences in gender or body frame. However, typically BMI of 25 or greater defines the point where the risk of disease increases due to excess weight. Assays for measuring energy expenditure, body composition and weight loss in animals that would be useful for determining effect of an antibody of the invention on an obese subject are known in the art, see e.g., International Patent Publication Number WO 01/87335 (incorporated herein by reference).

Hunger is a desire for food and is normal. Hunger typically occurs when caloric intake is less than caloric expenditure (negative energy balance) and in anticipation of an entrained meal even when the individual is in a positive energy balance. Hyperphagia and impaired satiety are defined as excessive ingestion of food beyond that needed for basic energy requirements. Ingestion may occupy unusual amounts of time. Eating may be obligatory and disrupt normal activity and can be symptomatic of various disorders. Hyperphagic or impaired satiety conditions may occur in association with central nervous system (CNS) disorders including gangliocytoma of the third ventricle, hypothalmic astrocytoma, Kleine-Levin Syndrome, Froehlich's Syndrome, Parkinson's Disease, genetic disorders including Praeder-Willi Syndrome (deletion on the long arm of chromosome 15), psychiatric disorders including anxiety, major depressive disorder, depressive phase of bipolar disorder, seasonal affective disorder, and schizophrenia, psychotropic medication, including delta-9 tetrahydrocannabinol, antidepressants and neuroleptics, may induce hyperphagia. Sleep disorders including sleep apnea is also associated with hyperphagia.

Type II diabetes mellitus, also called non-insulin dependent diabetes mellius (NIDDM), is present in subjects whose insulin their body is still capable of producing is not physiologically effective. An individual can be predisposed to NIDDM by both genetic and environmental factors. Heredity, obesity, and increased age play a major role in the onset of NIDDM. Risk factors include prolonged stress, sedentary lifestyle and certain medications affecting hormonal processes in the body. Eighty percent or more of the people with NIDDM are obese indicating obesity to be a predominant link to the development of NIDDM. An antibody of the invention may also be used to treat or prevent eating disorders including, but not limited to, bulimia, anorexia nervosa, binge eating and metabolic syndrome.

An antibody of the invention may be used to treat or prevent a subject, preferably a human, in need thereof for obesity, obesity-related disorders, NIDDM (Type II diabetes), Prader-Willi syndrome, eating disorders, hyperphagia, impaired satiety, anxiety, gastric motility disorders (including e.g., irritable bowel syndrome and functional dyspepsia), insulin resistance syndrome, metabolic syndrome, dyslipidemia, atherosclerosis, hypertension, hyperandrogenism, polycystic ovarian syndrome, cancer, and cardiovascular disorders.

The use of an anti-hGhrelin monoclonal antibody of the present invention for treating or preventing of at least one of the aforementioned disorders in which ghrelin (acylated or des-acyl or both) activity is detrimental is also contemplated herein. Additionally, the use of an anti-ghrelin monoclonal antibody of the present invention for use in the manufacture of a medicament for the treatment of at least one of the aforementioned disorders in which ghrelin activity is detrimental is contemplated.

As used herein, the terms “treatment”, “treating”, and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment”, as used herein, includes administration of a compound of the present invention for treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease or disorder or alleviating symptoms or complications thereof. Treatment may be in conjunction with behavior modification such as limitation of food intake and exercise. Treating obesity therefore includes inhibition of food intake, inhibition of weight gain, and/or inducing weight loss in subjects in need thereof.

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation.

The following examples are offered for illustrative purposes only, and are not intended to limit the scope of the present invention in any way.

### EXAMPLES

### Example 1

### Anti-Ghrelin Fab Synthesis

The CDR and framework sequences disclosed herein are identified from clones of Fab fragments isolated from antibody libraries generated from antibody RNA created by immunized C57/B16 wild-type mice using Omniclonal™ antibody technology (Biosite®, San Diego, Calif.). Amino acid sequences of isolated Fabs 3281, 4731 and 4281, are shown in Table 2 herein.

### Example 2

### Competitive ELISA Assay

Anti-hGhrelin Fabs of the invention are tested in a competitive ELISA assay, an assay in which a compound that might compete with an antigen for binding to a Fab is first combined with the Fab in solution phase. Then binding of the Fab to the antigen coated on a plate is measured.

Each well of a 96-well plate is coated with 60 μl BSA-hGhrelin antigen (i.e., BSA conjugated, full-length, acylated human ghrelin, 2 μg/ml in carbonate buffer, pH 9.6). The plate is incubated at 4° C. overnight. The wells are aspirated and washed twice with washing buffer (20 mM Tris-Cl, pH 7.4, 0.15 M NaCl, 0.1% Tween 20). Compounds (i.e., human ghrelin or ghrelin analogs or ghrelin fragments) are diluted into antibody solution. The antibody solution has a mouse anti-human ghrelin Fab. The compound concentration is varied from 0 to 5 μg/ml, but the Fab concentration is kept constant at 0.1 μg/ml in blocking solution (10 mg/ml BSA in wash buffer). After a 1-hour incubation at room temperature, 50 μl of compound-Fab solution is added to the BSA-hGhrelin coated wells in triplicate. The plates are incubated for 1 hour at room temperature. The wells are then washed three times with washing buffer.

Peroxidase-conjugated secondary antibody (50 μl goat anti-mouse kappa HRP (Southern Biotech), diluted 1:2000 in blocking buffer) is added to each well and incubated for 1 hour at room temperature. The wells are then washed 4 times with washing buffer. Fifty microliters of chromogenic substrate (i.e., OPD substrate) is added to each well and allowed to develop at room temperature for 10 minutes. The reaction is stopped by adding 100 μl 1N HCl to each well. The absorbance of the wells is read at 490 nm.

Ghrelin fragments of various lengths may be tested, e.g.,: (1) full length (amino acids 1-28), acylated or des-acyl human (or rat) ghrelin, (2) amino acids 20-28 of human (or rat) ghrelin, (3) amino acids 14-28 of human (or rat) ghrelin, (4) amino acids 1-27 of acylated or des-acyl, human (or rat) ghrelin, and (6) amino acids 18-28 of human (or rat) ghrelin. The average absorbance from triplicate wells is determined.

Antibodies of the invention bind the antigenic epitope residing within amino acids 14-27 of human or rat ghrelin regardless of whether the ghrelin is acylated or des-acyl.

### Example 3

### FLIPR In Vitro Activity Assay

The in vitro FLIPR®Calcium Assay system (Molecular Devices) is used with hamster AV12 cells stably transfected to express the GHS-R1a human ghrelin receptor. This assay evaluates changes in intracellular calcium as a means of detecting ghrelin/GHS-R1a binding and signaling in the presence or absence of a Fab of the invention. This functional assay is used to further map the location of the epitope to which the monoclonal antibodies of the invention bind.

AV12 cells are grown in growth media (DMEM/F12 (3:1), 5% fetal bovine serum, 50 μg/ml hygromycin and 50 μg/ml zeocin) to about 50-90×10⁶ cells per T-150 flask. The cells are then trypsinized, washed and distributed into Biocoat black poly-D-lysine coated plates (60,000 cells in 100 μl growth media per well). The cells are incubated for about 20 hours at 37° C. in 5% CO₂. The media is removed from the plate and 150 μl HBSS (Gibco 14025-037) is added to each well and then removed. Then dye is loaded into the cells by adding to each well 50 μl loading buffer [5 μM Fluo-4 μM (Molecular Devices), 0.05% Pluronic in FLIPR buffer [Hank's Balanced Salt with calcium (HBSS, Gibco 14025-092) and 0.75% BSA (Gibco)]. The plate is further incubated at 37° C. in 5% CO₂ for one hour. The wells are then washed twice with HBSS and 50 μL FLIPR buffer is then added per well.

Samples are prepared by combining 7.2 μl calcium concentrate (CaCl₂-2H₂O in water at 3.7 mg/ml mixed 1:1 with HBSS and filter sterilized) with 30 μl peptide, 30 μl Fab (of varying concentration), and 16.8 μl hGhrelin (2.5 μM stock) in 3.75% BSA/50% HBSS. The final concentration of the sample solution is 0.75% BSA, and calcium at approximately the same concentration as in the FLIPR buffer. Fifty microliters of the sample solution is added to the 50 μl FLIPR buffer in the well with the AV12 cells. The final concentration of the peptide is 100 nM and the final concentration of the hGhrelin is 0.83 nM. The cell plate is shaken for about 15 seconds prior to loading it into the FLIPR instrument. Test samples or control samples are added to each well, and read by a Fluorometric Imaging Plate Reader (Molecular Devices).

If there is no Fab or an irrelevant antibody present in the solution, the full-length hGhrelin will be free to bind the GHS-R1a receptor on the AV12 cells and signal transduction will occur resulting in comparatively high values in the assay. If a Fab is present that binds to the full-length hGhrelin in the solution, then the full-length hGhrelin binding to the GHS-R1a receptor is inhibited and signal transduction is thereby inhibited resulting in comparatively lower values in the assay. However, if a peptide (i.e., a fragment of human ghrelin) is also added to the solution and the Fab binds the peptide, then the full-length hGhrelin is not prevented from binding the GHS-R1a receptor, signal transduction is not inhibited, and the values in the assay are comparatively high. Conversely, if a peptide is added to the solution and the Fab does not bind the peptide, then the Fab will be available to bind the full-length hGhrelin in the solution and the values in the assay will be comparatively low. Notably, the peptide fragments tested are not active and will not bind the GHS-R1a receptor; therefore, their presence will not contribute to background levels. The peptides competing with hGhrelin for Fab binding were used in the assay at a concentration over 50 times that of hGhrelin. The Fab concentration used was determined by titration to be a level that will give approximately 95% inhibition of 1 nM hGhrelin activity.

### Example 4

### FLIPR Assay with Active Analogs

Active human ghrelin analogs or full-length, acylated rat ghrelin are combined with a Fab of the invention to determine if the Fab can inhibit the analog activity. This FLIPR Assay is performed substantially like that described in Example 3 herein, with the following exceptions. Analogs tested here are active and bind the GHS-1a receptor to which full-length acylated hGhrelin binds. Therefore, no full-length acylated hGhrelin is added to the sample in this assay.

The active analogs are used at a concentration that yields sub-maximal activity. The analogs are incubated with the Fab at concentrations known to fully inhibit 1 nM acylated hGhrelin.

Samples for this assay are prepared by combining 7.2 μl calcium concentrate (CaCl₂-2H₂O in water at 3.7 mg/ml mixed 1:1 with HBSS and filter sterilized) with 60 μl Fab (of varying concentration), and 16.8 μl peptide in 3.75% BSA/50% HBSS. The final concentration of the sample solution is 0.75% BSA, and the calcium concentration is approximately the same concentration as in the FLIPR buffer. Fifty microliters of the sample solution is added to the 50 μL FLIPR buffer in the well with the AV12 cells. Other aspects of the assay are the same as described in Example 3.

Resulting from Examples 3 and 4, human ghrelin peptides spanning amino acids 14-28 result in significant reduction of 3281, 4731, and 4281 Fab inhibition while the peptide spanning amino acids 4-20 has no reduction of inhibition of the Fabs. Additionally, ghrelin peptide 18-28 had no reduction of inhibition for Fab 3281. Fabs 3281, 4731, and 4281 bind to and inhibit the analog activity of human ghrelin 1-27. From this data, it is conclusive that the antigenic epitope resides within the peptide spanning amino acids 14-27 of human ghrelin that is identical in rat ghrelin.

### Example 5

### Affinity Measurement of Monoclonal Antibodies

The affinity (K_{D}) of anti-ghrelin Fab 3281, 4731 and 4281 are measured using a BIAcore™ 2000 instrument containing a CM5 sensor chip. The BIAcore™ utilizes the optical properties of surface plasmon resonance to detect alterations in protein concentration of interacting molecules within a dextran biosensor matrix. Except where noted, all reagents and materials are purchased from BIAcore™ AB (Upsala, Sweden). Measurements are performed at about 25° C. Samples containing rat or human ghrelin (full length, C8-acylated or des-acylated) are dissolved in HBS-EP buffer (150 mM sodium chloride, 3 mM EDTA, 0.005% (w/v) surfactant P-20, and 10 mM HEPES, pH 7.4). A capture antibody, goat anti-mouse Kappa (Southern Biotechnology, Inc), is immobilized onto flow cells using amine-coupling chemistry. Flow cells (1-4) are activated for 7 minutes with a 1:1 mixture of 0.1 M N-hydroxysuccinimide and 0.1 M 3-(N,N-dimethylamino)propyl-N-ethylcarbodiimide at a flow rate of 10 μl/min. Goat anti-mouse Kappa (30 μg/mL in 10 mM sodium acetate, pH 4.5) is manually injected over all 4 flow cells at a flow rate of 10 μL/min. The surface density is monitored and additional goat anti-mouse Kappa is injected if needed to individual cell until all flow cells reach a surface density of 4500-5000 response units (RU). Surfaces are blocked with a 7 minute injection of 1 M ethanolamine-HCl, pH 8.5 (10 μL/min). To ensure complete removal of any noncovalently bound goat anti-mouse Kappa, 15 μL of 10 mM glycine, pH 1.5 is injected twice. Running buffer used for kinetic experiments contained 10 mM HEPES, pH 7.4, 150 mM NaCl, 0.005% P20.

Collection of kinetic binding data is performed at maximum flow rate (100 μL/min) and a low surface density to minimize mass transport effects. Each analysis cycle consists of (i) capture of 300-350 RU of Fabs (BioSite) by injection of 5-10 μL of 5 μg/ml solution over flow cell 2, 3 and 4 for different Fabs at a flow rate of 10 μL/min., (ii) 200 μL injection (2 min) of hGhrelin (concentration range of 50 nM to 0.78 nM in 2-fold dilution increments) over all 4 flow cells with flow cell 1 as the reference flow cell, (iii) 20 min dissociation (buffer flow), (iv) regeneration of goat anti-mouse Kappa surface with a 15 sec injection of 10 mM glycine, pH 1.5, (v) a 30 sec blank injection of running buffer, and (vi) a 2 min stabilization time before start of next cycle. Signal is monitored as flow cell 2 minus flow cell 1, flow cell 3 minus flow cell 1 and flow cell 4 minus flow cell 1. Samples and a buffer blank are injected in duplicate in a random order. Data are processed using BLAevaluation v3.1 software and data are fit to a 1:1 binding model in either BIAevaluation v3.1 or CLAMP global analysis software. Values from representative experiments result in
(i) kₒₙ values between 8.64×10⁵ and 2.94×10⁶ (1/Msec), k_{off} values between 4.86×10⁻⁴ and 3.94×10⁻³ (1/sec), and K_{D} values between 4.36×10⁻⁹ and 8.62×10⁻¹¹ M for Fabs of the invention and acylated human ghrelin;
(ii) kₒₙ values between 1.6×10⁵ and 1.42×10⁶ (1/Msec), k_{off} values between 4.98×10⁴ and 2.72×10⁻³ (1/sec), and K_{D} values between 5.53×10⁻⁹ and 5.63×10⁻¹¹ (M) for Fabs of the invention and acylated rat ghrelin; and

(iii) kₒₙ values between x and y 1/Msec, koff values between x and y 1/sec, and K_{D} values between x and y M for Fabs of the invention and des-acyl human ghrelin.

TABLE 2
Anti-Ghrelin Fab Sequences

SEQ ID NO: 1 LCVR CDR1 Fab 3281
RSSQSLGHSNGNTYLH
SEQ ID NO: 2 LCVR CDR1 Fab 4731, 4281
RSSQSLVHSNGNTYLH
SEQ ID NO: 3 LCVR CDR1 Consensus
RSSQSLX₇HSNGNTYLH
X₇ is G(gly), A(ala), V(val), L(leu) or I(ile)
SEQ ID NO: 4 LCVR CDR2 Fabs 3281, 4731 and 4281
KVSNRFS
SEQ ID NO: 5 LCVR CDR3 Fabs 3281, 4731 and 4281
SQSTLVPWT
SEQ ID NO: 6 HCVR CDR1 Fabs 3281 and 4731
GYTFTSYWMH
SEQ ID NO: 7 HCVR CDR1 Fab 4281
GYTFTSYWIH
SEQ ID NO: 8 HCVR CDR1 Consensus
GYTFTSYWX₉H
X₉ is M(met), I(ile), L(leu) or V(val)
SEQ ID NO: 9 HCVR CDR2 Fabs 3281 and 4731
YINPSTGYTEYTQKFKD
SEQ ID NO: 10 HCVR CDR2 Fab 4281
YIDPGIGNIEYNQKFQD
SEQ ID NO: 11 HCVR CDR2 consensus
YIX₃PX₅X₆GX₈X₉IEYX₁₃QKFX₁₇D
X₃ is N(asn), Q(gln), D(asp) or E(Glu)
X₅ is S(ser), T(thr), G(gly) or A(ala)
X₆ is T(thr), S(ser), I(ile), L(leu) or V(val)
X₈ is Y(tyr), N(asn) or Q(gln)
X₉ is T(thr), S(ser), I(ile), L(leu) or V(val)
X₁₃ is T(thr), S(ser), N(asn) or Q(gln)
X₁₇ is K(lys), R(arg), N(asn) or Q(gln)
SEQ ID NO: 12 HCVR CDR3 Fabs 3281, 4731 and 4281
DGYDEDY
SEQ ID NO: 13 LCVR Fab 3281
STPAWADAVMTQIPLTLSVTIGQPASISb RSSQSLGHSNGNTYLHWYLQK
PGQSPKLLIYKVSNRFSGVPDRFSGSGSGTDFTLKISRVEAEDLGTYFCS
QSTLVPWTFGGGTKLEIKRADAAPTV
SEQ ID NO: 14 LCVR Fabs 4731 and 4281
STPAWADVVMTQTPLSLPVSLGDQASISCRSSQSLVHSNGNTYLHWYLQK
PGQSPKLLIYKVSNRFSGVPDRFSGSGSGTDFTLKISRVEAEDLGTYFCS
QSTLVPWTFGGGTKLEIKRADAAPTV
SEQ ID NO: 15 HCVR Fabs 3281 and 4731
QVQLQQSRAELAKPGASVKMSCKASGYTFTSYMWHWVKQGPGQGLEWIGY
INPSTGYTEYTQKFKDKATLTADKSSSTAYMQLSSLTSEDSAVYYCATDG
YDEDYWGQGTTLTVSSAKTTPP
SEQ ID NO: 16 HCVR Fab 4281
QVQLQQSRAELAKPGASVKMSCKASGYTFTSYWIHWIKQRPGQGLEWIGY
IDPGIGNIEYNQKFQDKATLTADKSSSIVYMQLNRLTSEDSAVYYCATDG
YDEDYWGQGTTLTVSSAKTTPP
SEQ ID NO: 17 4731 LCVR polynucleotide
TCTACTCCAGCTTGGGCAGATGTTGTGATGACCCAAACTCCACTCTCCCT
GCCTGTCAGTCTTGGAGATCAAGCCTCCATCTCTTGCAGATCTAGTCAGA
GCCTTGTACACAGTAATGGAAACACCTATTTACATTGGTACCTGCAGAAG
CCAGGCCAGTCTCCAAAGCTCCTGATCTACAAAGTTTCCAACCGATTTTC
TGGGGTCCCAGACAGGTTCAGTGGCAGTGGATCAGGGACAGATTTCACAC
TCAAGATCAGCAGAGTGGAGGCTGAGGATCTGGGAACTTATTTCTGCTCT
CAAAGTACACTTGTTCCGTGGACGTTCGGTGGAGGCACCAAGCTGGAAAT
CAAGCGGGCTGATGCTGCACCAACTGTA
SEQ ID NO: 18 4731 HCVR polynucleotide
caggtccagctgcagcagtctagggctgaactggcaaaacctggggcctc
agtgaagatgtcctgcaaggcttctggctacacctttactagctactgga
tgcactgggtaaaacaggggcctggacagggtctggaatggattggatac
attaatcctagcactggttatactgagtacactcagaagttcaaggacaa
ggccacattgactgcagacaaatcctccagcacagcctacatgcaactga
gcagcctgacatctgaggactctgcagtctattactgtgcaacagatggt
tacgacgaggactactggggccaaggcaccactctcacagtctcctcagc
caaaacgacaccccca
SEQ ID NO: 19 Human ghrelin
GSSFLSPEHQRVQQRKESKKPPAKLQPX₂₈
wherein X₂₈ is Arg(R) or absent
SEQ ID NO: 20 amino acids 14-27 of human ghrelin
QRKESKKPPAKLQP

1-42. (canceled)

43. A monoclonal antibody or antigen-binding portion thereof,
wherein the light chain variable region and heavy chain variable region are selected from the group consisting of:
(a) a light chain variable region comprising a peptide having the amino acid sequence shown in SEQ ID NO: 13 and a heavy chain variable region comprising a peptide having the amino acid sequence shown in SEQ ID NO: 15;
(b) a light chain variable region comprising a peptide having the amino acid sequence shown in SEQ ID NO: 14 and a heavy chain variable region comprising a peptide having the amino acid sequence shown in SEQ ID NO: 15; and
(c) a light chain variable region comprising a peptide having the amino acid sequence shown in SEQ ID NO: 14 and a heavy chain variable region comprising a peptide having the amino acid sequence shown in SEQ ID NO: 16.

44. The monoclonal antibody or antigen-binding portion thereof of claim 43, comprising a heavy chain constant region selected from the group consisting of IgG₁, IgG₂, IgG₃, IgG₄, IgA, IgE, IgM, and IgD.

45. The monoclonal antibody or antigen-binding portion thereof of claim 43, comprising a kappa or lambda light chain constant region.

46. The monoclonal antibody or antigen-binding portion thereof of claim 43, which is a Fab fragment, a F(ab′)₂ fragment, or a single chain Fv fragment.

47. The monoclonal antibody or antigen-binding portion thereof of claim 43, which is chimeric.

48. A pharmaceutical composition, comprising said monoclonal antibody or antigen-binding portion thereof of claim 43, and a pharmaceutically acceptable carrier, diluent, or excipient.

49. A method of treating obesity or a related disorder in a mammal, comprising administering to a patient in need thereof an effective amount of said monoclonal antibody or antigen-binding portion thereof of claim 43.

50. The method of claim 49, wherein said related disorder is selected from the group consisting of NIDDM, Prader-Willi syndrome, an eating disorder, hyperphagia, impaired satiety, anxiety, and a gastric motility disorder.

51. A method of inhibiting ghrelin activity or decreasing active ghrelin levels in a subject, comprising administering to said subject an effective amount of a monoclonal antibody or antigen-binding portion thereof of claim 43.

52. The method of claim 49, wherein said mammal is a human.

53. The method of claim 50, wherein said mammal is a human.

54. The method of claim 51, wherein said subject is a human.
